(19) United States
(12) Patent Application Publication
(10) Pub. No.: US 2006/0148064 A1
(43) Pub. Date: Jul. 6, 2006

(54) Apparatus and method for immunotherapy of a cancer through controlled cell lysis

(76) Inventor: Pramod K. Srivastava, Avon, CT (US)

Correspondence Address: JONES DAY, 222 EAST 41ST ST, NEW YORK, NY, 10017, US

(21) Appl. No.: 10/532,660

(22) PCT Filed: Oct. 27, 2003
(86) PCT No.: PCT/US03/34283

§ 371 Date: Jan. 27, 2006

Related U.S. Application Data
(60) Provisional application No. 60/421,394, filed on Oct. 25, 2002.

(51) Int. Cl. C12M 1/34

(52) U.S. Cl. 435/287.2

(57) ABSTRACT

The treatment device includes an extraction mechanism, configured to extract a tissue sample from a patient or a tumor, coupled to a lysis mechanism. The lysis mechanism is configured to induce lysis of the tissue sample into a lysed tissue sample. The treatment device also includes an administration mechanism that is coupled to both the extraction mechanism and the lysis mechanism. Further included with the treatment device is an additive mechanism configured to add an additive solution to the lysed tissue sample before the lysed tissue sample is administered to the patient. Furthermore, the invention also provided a method for treating a cancer. The method includes the steps of extracting a tissue sample from a patient or a tumor into a chamber of a treatment device. Lysing the tissue sample into an laysed tissue sample within the chamber of the treatment device and administering the lysed tissue sample directly from the treatment device into the patient.

### 1. INTRODUCTION

Generally this invention relates to a device and method for treating or preventing disease or cancer. More specifically, this invention relates to a device and method for making an autologous lysed tissue sample obtained by controlled cell lysis. The lysed tissue sample made by this invention can elicit an immune response, prevent, or treat disease or cancer.

### 2. BACKGROUND OF THE INVENTION

Cancer effects millions of people and results in many deaths each year. To address this, a tremendous amount of resources are spent on cancer research each year. However, despite the vast resources invested, cures or successful treatments for cancer are rare.

Cancer is a pathological condition characterized by the proliferation of malignant neoplasms (tumors) that tend to invade surrounding tissue. There are primarily two characteristics of cancer that allow it to avoid immune system recognition and avoid eliciting an immune response. First, cancer cells are normal host cells that become mutated and proliferate in a non controlled manner forming tumors. The immune system, which typically rids the body of invading, infectious and diseased matter does recognize cancerous tissue, however, for unknown reasons this recognition does not always translate into a response sufficient to eliminate the disease. Thus, cancer can severely degrade the quality and longevity of the infected individual. Second, tumors have the ability to create an immunosuppressive environment. Therefore, even if the immune system recognizes the cancer cells as foreign or diseased, the immunosuppressive environment created by the tumor may keep the immune system suppressed around the tumor.

Currently there are several main cancer treatments in use, namely, chemotherapy, radiation, surgery, and immunotherapy.

Chemotherapy and radiation generally do not differentiate, but do to some extent, between cancer cells and normal tissue cells. Therefore, in use, the radiation and chemotherapy that is used to kill the cancer cells also kills healthy normal cells.

Surgery, on the other hand, is directed at removing the cancerous tissue. However, it is very difficult to surgically remove all the cancerous tissue because it becomes embedded within the surrounding tissue. Furthermore, it is not possible to operate in some areas of the body, such as areas of the brain.

Finally, immunotherapy uses the host immune system to fight cancer. This is done by introducing specific substances associated with cancer cells to the immune system so that the immune system recognizes the cancerous cells and mounts an attack against the cancer cells.

Immunotherapy utilizes the immune system to rid the body of disease and infection. Immune system cells actively scan their environments with surface receptor proteins, called antibodies. Antibodies recognize and distinguish between native host cells and foreign matter, whether the foreign matter is an inert particle or a living pathogenic microorganism such as a bacterium or virus. Once the immune system antibodies recognize invading foreign matter or an antigen, the immune system mounts a specific attack against that foreign matter or antigen. This attack consists of the proliferation of more immune system cells which secrete the specific antibody that has affinity for the previously recognized foreign matter or antigen. Next, the system attempts to rid the body of the foreign matter or antigen by either killing it or by recruitment of cells which engulf it.

The immune system may also be stimulated by cellular necrosis or lysis, hereinafter referred to as lysis or lytic cell death. Lysis is the injury, destruction, or death of cells that results in spillage of the intracellular components. Majno, G., Joris, I., Apoptosis, Oncosis, and Necrosis: An Overview of Cell Death, Am J Pathol 1995; 146: 3-15. Upon lysis an immune response is initiated by the dendritic cells (DCs) and macrophages of the immune system. Gallucci, S., Lolkema, M., Matzinger, P., Natural Adjuvants: Endogenous Activators of Dendritic Cells, Nat Med 1999; 5: 1249-55; Sauter, B., Albert, M. L., Francisco, L., Larsson, M., Somersan, S., Bhardwaj, N., Consequences of Cell Death: Exposure to Necrotic Tumor Cells, But Not Primary Tissue Cells or Apoptotic Cells, Induces the Maturation of Immunostimulatory Dendritic Cells, J Exp Med 2000; 191: 423-34; and Basu, S., Binder, R., Suto, R., Anderson, K. M., Srivastava, P. K., Necrotic But Not Apoptotic Cell Death Releases Heat Shock Proteins, Which Deliver A Partial Maturation Signal to Dendritic Cells and Activate the NF-kappa B Pathway, Int Immunol 2000; 12: 1539-46. DCs bear receptors for heat shock proteins released during lysis of cancer cells. Gallucci, S., Lolkema, M., Matzinger, P., Natural Adjuvants: Endogenous Activators of Dendritic Cells, Nat Med 1999; 5: 1249-55; Sauter, B., Albert, M. L., Francisco, L., Larsson, M., Somersan, S., Bhardwaj, N., Consequences of Cell Death: Exposure to Necrotic Tumor Cells, But Not Primary Tissue Cells or Apoptotic Cells, Induces the Maturation of Immunostimulatory Dendritic Cells, J Exp Med 2000; 191: 423-34; Basu, S., Binder, R., Suto, R., Anderson, K. M., Srivastava, P. K., Necrotic But Not Apoptotic Cell Death Releases Heat Shock Proteins, Which Deliver A Partial Maturation Signal to Dendritic Cells and Activate the NF-kappa B Pathway, Int Immunol 2000; 12: 1539-46; Somersan et al., Primary Tumor Tissue Lysates Are Enriched in Heat Shock Proteins and Induce the Maturation of Human Dendritic Cells, J Immunol 2001; 167: 4844-52; and Basu et al., CD91 Is a Common Receptor for Heat Shock Proteins gp96, hsp90, hsp70, and Calreticulin, Immunity 2001; 14: 303-313. Engagement of the heat shock proteins on DCs stimulate the release of cytokines and chemokines. Basu, S., Binder, R., Suto, R., Anderson, K. M., Srivastava, P. K., Necrotic But Not Apoptotic Cell Death Releases Heat Shock Proteins, Which Deliver A Partial Maturation Signal to Dendritic Cells and Activate the NF-kappa B Pathway, Int Immunol 2000; 12: 1539-46; Panjwani et al., Heat Shock Proteins gp96 and hsp70 Activate the Release of Nitric Oxide by APC's, J. Immunol 2002; 168: 2997-3003. Chemokines and cytokines are responsible for further mobilization of the immune system. DCs also begin to show changes characteristic of maturation and migration to the draining lymph nodes. Basu, S., Binder, R., Suto, R., Anderson, K. M., Srivastava, P. K., Necrotic But Not Apoptotic Cell Death Releases Heat Shock Proteins, Which Deliver A Partial Maturation Signal to Dendritic Cells and Activate the NF-kappa B Pathway, Int Immunol 2000; 12: 1539-46; Binder et al., Heat Shock Protein gp96 Induces Maturation and Migration of CD11c⁺ Cells in Vivo, J. Immunol 2000; 165: 6029-6035. All of the foregoing references are incorporated herein by reference in their entireties.

Tumor cell lysis also mediates another powerful reaction that brings the adaptive immune system into play. Lytic cell death releases heat shock protein-peptide complexes that are taken up by the DCs. Subsequently, the peptide complexes are re-presented on the surface of DCs to stimulate CD8+ and CD4+ T cells, respectively. Binder, R. J., Han, D. K., Srivastava, P. K., CD91: A Receptor for Heat Shock Protein gp96, Nat Immunol 2000; 1: 151-5. Therefore, cellular lysis engages all the major components of the immune system.

Attempts at using immunotherapy, and specifically cell lysis, to treat cancer began as early as 1777. Researchers began to develop cancer vaccines derived from neoplastic tissue samples. They began inoculating themselves and others with compositions comprising cancer tissue, extracts from cancer tissue, cultured cancer cells, and tumor cells modified by viral infection, enzymatic digestion, or chemical treatment (Oettgen, H. F., and Old, L. J. 1991, The History of Cancer Immunotherapy, in Introduction to the Biologic Therapy of Cancer, DeVitta, V. T., Hellman, S., and Rosenberg, S. A. Editors, Lippincott, Philadelphia, pp. 87-119).

Over the years immunotherapy methods were refined. Techniques such as fractionalization (the purification of selected proteins from the remaining intracellular components) were developed to extract the intracellular agents thought to be responsible for the immune system stimulus following cell lysis. In 1970, Hughes et al. reported the use of homogenized, fractionated tumor tissue as a vaccine for clinical cancer immunotherapy (Hughes, L. E. et al., A Study in Clinical Cancer Immunotherapy, 1970, Cancer, 26(2): 269-78). The techniques used to create this vaccine include the steps of homogenizing autologous isolated tumor samples and then breaking the isolated cells by sonication. The lysed material is subjected to multiple centrifuging steps at high speed for up to 45 minutes. Portions of the fractionated matter are then used as a treatment.

Another technique using tumor extracts for treatment as a vaccination is disclosed by Humphrey L. J. et al., Adjuvant Immunotherapy for Melanoma, Journal of Surgical Oncology, 25: 303-05. This technique homogenizes tumor tissue and centrifuges for up to 74 minutes. This technique also treats the sample with a solution and filters the supernatant to concentrate the vaccine.

Cassel et al. describes using a virus to lyse tumor cells and then centrifuge and filter the fractionate to create a vaccine (Cassel, W. A. et al., Viral Oncolysate in the Management of Malignant Melanoma, 1977, Cancer 40: 672-79).

Yet another technique that uses an unfractionated method for vaccine creation is described in WO 02/30434 A1 to Srivastava, published Apr. 18, 2002, which is incorporated herein by reference. Srivastava discloses a method for the prevention and treatment of primary and metastatic neoplastic diseases and infectious diseases with compositions comprising unfractionated cellular proteins. These unfractionated cellular proteins are obtained by lysing cells and then centrifuging the lysate. Although the above references describe many different techniques for developing vaccines from cancer or other diseased tissue, autologous or allogeneic, they have several common drawbacks. For example, each technique involves many complicated steps that require expensive lab equipment and experienced lab technicians.

One particular drawback of the above described techniques is the time that is required to create the vaccine. Assuming an autologous vaccine is required or preferred, a sample of tissue must be obtained from the patient and then sent to a properly equipped lab with trained technicians to process the tissue sample and create the vaccine. These steps include lysing cells, centrifuging, fractionalizing, filtering, and clarifying the composition, treating the composition with different solutions; and the like. Next the vaccine must be transported back to the hospital or clinic for administration to the patient. This may require the patient to make several appointments and several visits to the clinic or hospital, thereby increasing the patients hardships including increased pain, greater travel expenses, lost time, and increased hospital expenses.

Another drawback is the likelihood of contamination. Because the above described techniques for making the vaccines require multiple steps the danger of contamination is high. The techniques require transporting the tissue and composition, as well as transferring the composition between containers and lab equipment. Each transfer of the composition increases the chance of contamination.

In light of the above, an apparatus and method that addresses the above drawbacks would be highly desirable. Specifically, a simple device that can extract a tissue sample, generate a vaccine, and administer the vaccine in a short amount of time, with a minimal amount of steps would be highly desirable.

### 3. SUMMARY OF THE INVENTION

According to the invention there is provided a treatment device for making a lysed tissue sample (vaccine) and treating disease. The treatment device has an extraction mechanism configured to extract a tissue sample from a patient or from a surgically removed tumor. The extraction mechanism can be either a collection needle coupled to a syringe having a chamber or a biopsy device including a stylet and cannula. Coupled to the extraction mechanism is a lysis mechanism. The lysis mechanism is configured to induce lysis of the tissue sample into a lysed tissue sample. The lysis mechanism is selected from a group consisting of: a pair of rotatable cylinders, a pair of intermeshing rotatable gears, a grate, a tortuous path, rotatable blades, a cooling mechanism, a heat exchanger, an ultrasonic mechanism, an ultrasonic probe, and any combination of the aforementioned. The treatment device also includes an administration mechanism that is coupled to both the extraction mechanism and the lysis mechanism. The administration mechanism comprises an administration needle in fluid communication with the lysed tissue sample and is configured to administer the lysed tissue sample to the patient.

The treatment device also includes an additive mechanism configured to add an additive solution to the lysed tissue sample before the lysed tissue sample is administered to the patient. The additive mechanism comprises a syringe in fluid communication with the extraction mechanism and is configured to hold an additive solution.

Also according to the invention there is provided a treatment device that includes an extraction mechanism configured to extract a tissue sample from a patient or a surgically removed tumor. The extraction mechanism includes a collection needle coupled to a chamber. The extraction mechanism can include a plunger configured to alter a pressure within a chamber of the extraction mechanism so as to extract the tissue sample and administer the lysed tissue sample. Disposed within the chamber is a lysis mechanism that is configured to induce lysis of the tissue sample into a lysed tissue sample. An additive mechanism is in fluid communication with a chamber of the extraction mechanism, and configured to add an additive solution to the lysed tissue sample before the lysed tissue sample is administered to the patient. Coupled with both the extraction mechanism and the lysis mechanism is an administration mechanism configured to administer the lysed tissue sample to the patient.

Also according to the invention there is provided a treatment device that includes an extraction mechanism configured to extract a tissue sample from a patient or a surgically removed tumor, where the extraction mechanism includes a syringe type device having a chamber coupled to a collection needle. A lysis mechanism is disposed within the chamber, and is configured to induce lysis of the tissue sample into a lysed tissue sample. An administration mechanism is coupled to both the extraction mechanism and the lysis mechanism, and is configured to administer the lysed tissue sample to the patient. Further, the administration mechanism includes a syringe type device having a chamber coupled to an administration needle.

According to the invention there is provided, within the chamber a tissue sample that can be from the group comprising a tumor sample; a tumor sample of a human, a tumor sample of an animal other than a human; a tumor sample that has been lysed and mixed with a fluid and homogenized; a tumor sample of a human that has been lysed and mixed with a fluid and homogenized; a tumor sample of an animal other than a human that has been lysed and mixed with a fluid and homogenized; an infected cell sample; an infected cell sample of a human; an infected cell sample of an animal other than a human; an infected cell sample that has been lysed and mixed with a fluid and homogenized; an infected cell sample of a human that has been lysed and mixed with a fluid and homogenized; and an infected cell sample of an animal other than a human that has been lysed and mixed with a fluid and homogenized, or any combination of the aforementioned.

According to the invention there is also provided a method for treating a cancer. The method includes the steps of: extracting a tissue sample from a cancerous area of a patient or from a surgically removed tumor into a chamber of a treatment device; lysing the tissue sample into a lysed tissue sample within the chamber of the treatment device; and administering the lysed tissue sample directly from the treatment device into the patient.

According to the invention there is provided one embodiment of the invention where, before extracting the tumor or tissue sample, a collection needle is attached to the chamber. The collection needle is then inserted into a tumor core of the patient or a surgically removed tumor before the extracting of the tissue sample is done.

According to the invention there is provided one embodiment where the lysing comprises, cooling the tissue sample to at least −196 degree Celsius for between five seconds to ten minutes and then warming the tissue sample to between 32-42 degrees Celsius and preferably 37 degrees Celsius for between five seconds to ten minutes.

The method for treating a human cancer also can include the step of adding an additive solution to the lysed tissue sample before the administering of the lysed tissue sample to the patient.

According to the invention there is one embodiment where, before administering the lysed tissue sample the collection needle is replaced with an administration needle.

According to the invention there is one embodiment where, before administering the lysed tissue sample, the administration needle of the treatment device is inserted into the patient at a different location to where the tissue sample extracting occurred.

The treatment device and method of the present invention addresses the drawbacks associated with the prior art by creating a lysed tissue sample in a simple, timely, and less expensive format than current techniques. What is more, the invention is safer and more convenient for the patient than the current techniques. The treatment device of the invention produces a lysed tissue sample within a treatment device without the current concerns regarding contamination. This treatment device can be either reusable and capable of sterilization by such techniques as autoclaving or the treatment device can be disposable, thereby eliminating any chance of cross contamination between patients. In addition, this treatment device can also be used right in the operating room or clinical exam room, making the procedure an outpatient procedure. Also, there is no expensive lab equipment required to make the lysed tissue sample, therefore saving money in the production stage of a lysed tissue sample. Furthermore, there are no complicated and time wasting steps of clarifying, fractionating, or purifying components of the lysed tissue sample.

According to yet another embodiment of the present invention, a kit for a device for producing an immune response is provided. The kit includes a lysis mechanism configured to induce cell lysis of a tissue sample and an administration mechanism coupled to the lysis mechanism, wherein the administration mechanism is configured to administer the lysed tissue sample to a patient. Furthermore, the kit includes instructions for using the device.

In another embodiment, the kit further includes an extraction mechanism configured to extract tissue from a subject. The extraction mechanism can include a collection needle, a biopsy needle, a stylet, or a cannula. The kit can also include an administration mechanism such as an admininistration needle. Further included in the kit can be a biologically active additive, a buffer, and/or a tissue sample.

### 4. BRIEF DESCRIPTION OF THE DRAWINGS

For a better understanding of the function and objects of the present invention reference should be made to the detailed description in conjunction with the accompanying drawings, in which:

FIG. 1 is a diagrammatic plan view of a treatment device for treating disease or cancer by eliciting an immune response, according to an embodiment of the invention;

FIG. 2A is a diagrammatic plan view of a lysis mechanism shown in FIG. 1, according to an embodiment of the invention;

FIG. 2B is a cross sectional view of the lysis mechanism shown in FIG. 1, as viewed along line X-X′ of FIG. 2A;

FIG. 2C is a diagrammatic plan view of another embodiment of the lysis mechanism shown in FIG. 1, according to another embodiment of the invention;

FIG. 2D is a diagrammatic plan view of yet another embodiment of the lysis mechanism shown in FIG. 1, according to yet another embodiment of the invention;

FIG. 2E is a cross sectional view of even another lysis mechanism, as viewed along line X-X′ of FIG. 1, according to even another embodiment of the invention;

FIG. 2F is a diagrammatic plan view of another embodiment of the lysis mechanism shown in FIG. 1, according to another embodiment of the invention;

FIG. 2G is a diagrammatic plan view of yet another embodiment of the lysis mechanism, according to yet another embodiment of the invention;

FIG. 2H is a diagrammatic plan view of still another embodiment of the lysis mechanism, according to still another embodiment of the invention;

FIG. 2I is a diagrammatic plan view of one other embodiment of the lysis mechanism, according to one other embodiment of the invention;

FIG. 2J is a diagrammatic plan view of one other embodiment of the lysis mechanism, according to one other embodiment of the invention; and

FIG. 3 is a flow chart for a method of producing and administering a lysed tissue sample for a disease or cancer by using the self contained treatment device of the present invention.

Like reference numerals refer to corresponding parts throughout the several views of the drawings. For ease of reference, the first number of any reference numeral generally indicates the figure number in which the reference numeral can be found.

### 5. DETAILED DESCRIPTION OF THE PREFERRED EMBODIMENT

There are multiple different therapies that may be used with the device of the present invention. The present invention is also useful for the prevention and treatment of with multiple different diseases, including cancer, infectious diseases, and immune disorders, e.g., infections, immunosuppressant, and immunostimulatory conditions. In addition, there are of the present invention. Some of the different therapies and disorders which the device and methods of the present invention are useful in treating are described below. Combination therapy encompasses, in addition to the treatment with the device and methods of the present invention, the uses of one or more modalities that aid in the prevention or treatment of infectious diseases, which modalities include, but are not limited to antibiotics, antivirals, antiprotozoal compounds, antifungal compounds, and antihelminthics. Other treatment modalities that can be used to treat or prevent infectious diseases include immunotherapeutics, polynucleotides, antibodies, cytokines, and hormones as described above.

### 5.1. Controlled Cell Lysis Apparatus

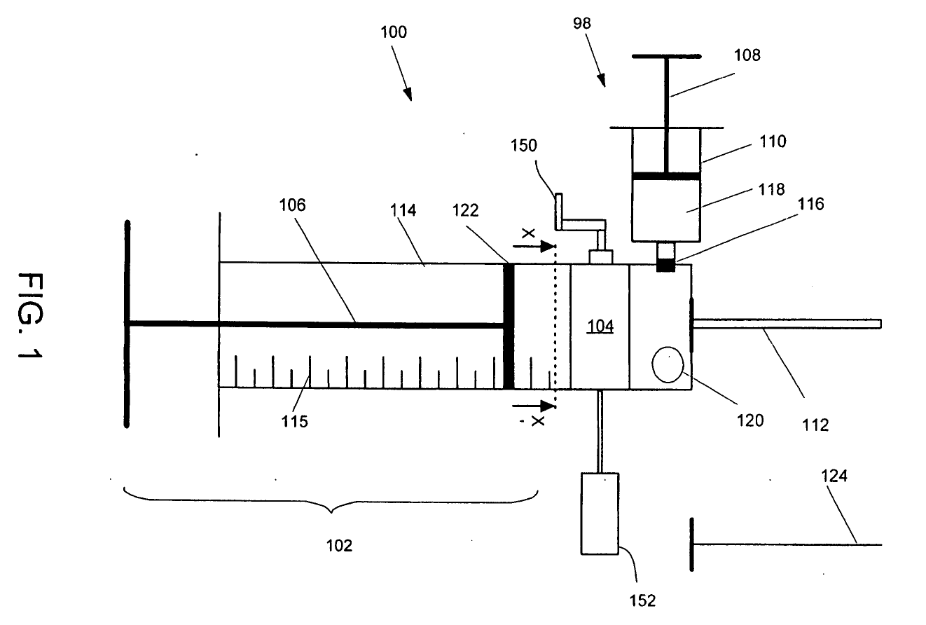

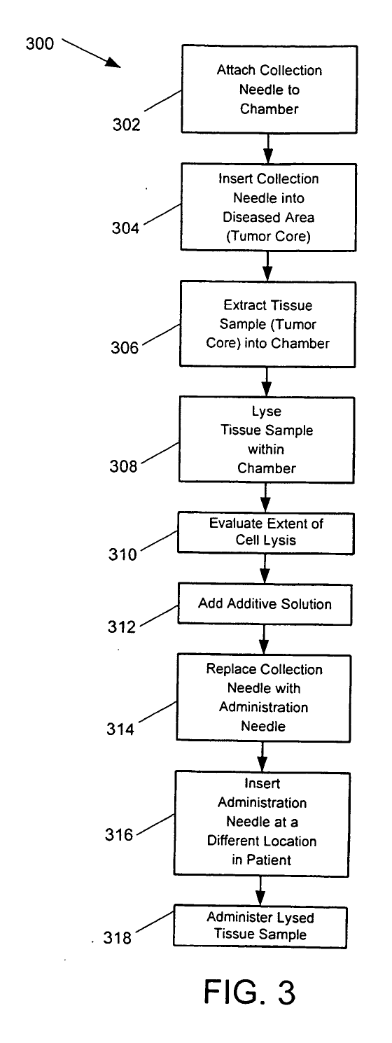

FIG. 1 is a diagrammatic plan view of a treatment device 100 for treating disease or cancer by eliciting an immune response. In a preferred embodiment, the treatment device 100 extracts a tissue sample (preferably a tumor core) from a patient or from a surgically removed tumor, causes or induces lysis of the tissue sample to produce a lysed tissue sample, such as an immunotherapy vaccine, mixes the lysed tissue sample with an additive solution, and reinserts or administers the lysed tissue sample into a patient. Treatment device 100 is suitable for use with, for example but not limited to: humans and animals other than humans such as primates, domestic animals like dogs and cats, and other animals such as rats, mice, birds, rabbits, guinea pigs, hamsters, and farm animals such as horses, cows, pigs, goats, or the like. Details of an exemplary method of using the treatment device 100 can be found below in relation to FIG. 3.

Treatment device 100 preferably is sterile and includes an extraction mechanism for extracting a tissue sample from a patient or from surgically removed tissue; a lysis mechanism for causing cell lysis of the tissue sample (i.e., lysis of the plasma membranes and preferably also intracellular membranes, thus releasing the contents of the cell); an additive mechanism for adding any additive solutions to the tissue sample either before, after, and/or concurrently with cell lysis; and an administration mechanism for reintroducing the lysed tissue sample into the patient. It should be stressed that all of these mechanisms form part of a single treatment device (even though the treatment device may be disassembled into parts). It should also be stressed that the treatment device may be constructed from: (1) disposable materials, such as plastic or the like, which can be disposed of after use; or (2) may be constructed from reusable materials, such as stainless steel or the like, which can be sterilized after each use by such techniques as autoclaving or the like.

The extraction mechanism preferably consists of a collection needle 112 coupled to a chamber 114 of a syringe type device 102 having a plunger 106 or the like. The chamber 114 preferably is made from an optically transparent material. Alternatively, a portion of the chamber 114 may have an optically transparent section, such as a viewing window. The chamber 114 also preferably has graduated volume markings 115 which represent the volume of a sample contained within the chamber 114. The units of the graduated volume markings 115 are preferably in microliters from approximately 10-100 microliters. The graduated volume markings 115 are inscribed on the wall of chamber 114 such that the contents of the chamber 114 can be viewed and estimated for determining the dosage of a lysed tissue sample to be administered to a patient, as described below with respect to FIG. 3. In use, chamber 114 also typically contains a solution, such as a saline solution, to facilitate lysis of the tissue sample and administration of the lysed tissue sample, as described below. Suitable solutions include, but are not limited to, a saline solution, a saline solution containing a surfactant such as Tween® 80 (polyoxyethylene sorbitan monooleate) or Tween® 20 (polyoxyethylene sorbitan monolaurate) made by Huanan Chemical and Industrial Corp., China or a saline solution containing sugars such as glycerol or polyethylene glycol (PEG). Such solutions preferably facilitate lysis of the tissue sample, minimize adsorption of proteins to the surfaces of the treatment device 100, facilitate administration of the lysed tissue sample, and are sterile. A suitable syringe type device 102 is the BREEZE® vacuum biopsy system made by Allegiance Healthcare Corp., McGaw Park, Ill.

The collection needle 112 is preferably a fine aspiration needle. A suitable example of a fine aspiration needle is the FNA made by Allegiance Healthcare Corp. McGaw Park, Ill. In use, the collection needle 112 is inserted into a patient, at or near an infected, diseased, or cancerous location, and a tissue or tumor sample is withdrawn through the collection needle 112 into the chamber 114 of the syringe type device 102. In a preferred use, the collection needle 112 is inserted into a tumor core, and a sample of the tumor core is extracted into the chamber 114 of the syringe type device 102. The tissue or tissue sample is then preferably used to create a lysed tissue sample for administration. A fresh tissue sample may be extracted each time it is desired to treat a patient such as once daily, once a week, every two weeks, once a month, or by another schedule as determined by a treating physician. However, in other embodiments, the sample may be stored in the treatment device 100 for later use or repetitive use at periodic intervals according to the schedule listed above.

In the embodiment where the collection needle 112 is a fine aspiration needle, extraction of the tissue sample is performed by retracting the plunger 106 of the syringe type device 102 to create a vacuum within the chamber 114. The vacuum created within the chamber 114 draws the tissue sample through the collection needle 112 and into the chamber 114.

In an alternative embodiment, the extraction mechanism is a biopsy device 102, such as the TRU-CUT® biopsy device made by Allegiance Healthcare Corp., McGaw Park, Ill. In this embodiment, the biopsy device 102 includes a collection needle 112 such as a stylet and cannula that is inserted into the patient to retrieve a tissue sample from an infected or diseased site. An example of a suitable collection needle, for this embodiment, is the PRESET™ Core Biopsy Needle made by INRAD Inc., Kentwood, Mich. In use, the stylet and cannula are inserted into a patient and an appropriate tissue sample is collected in the chamber 114.

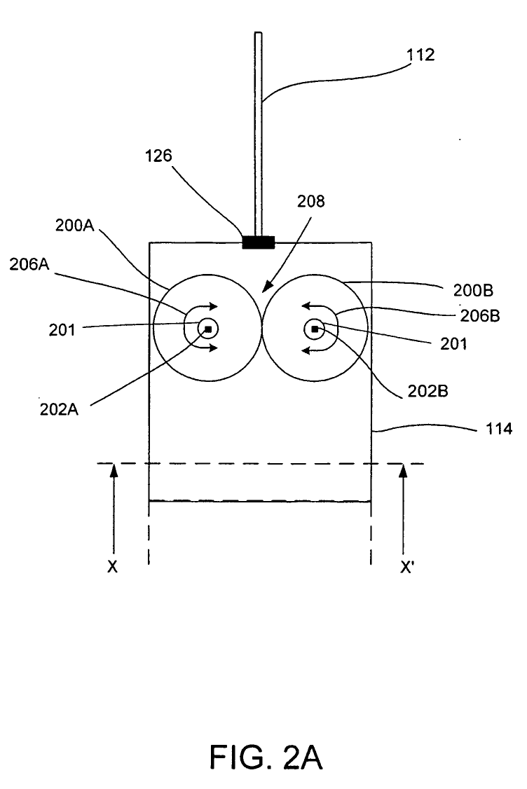

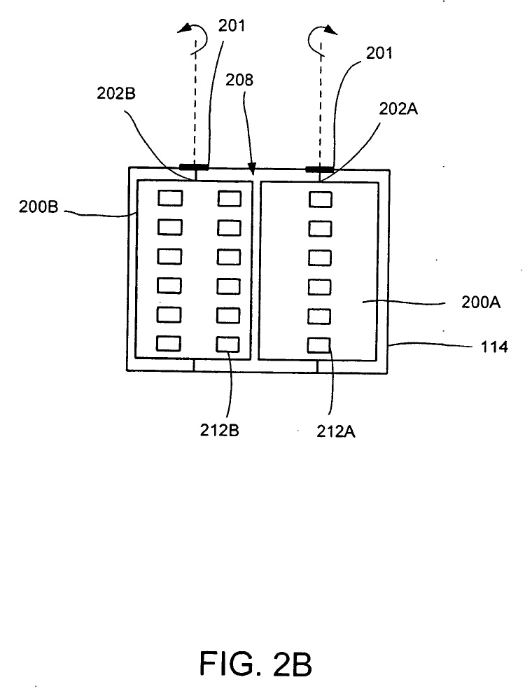

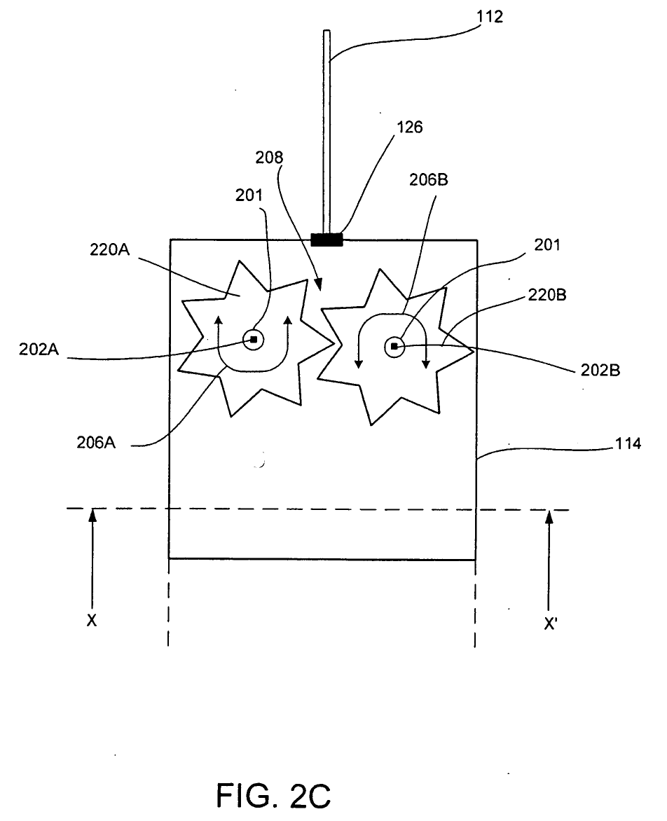

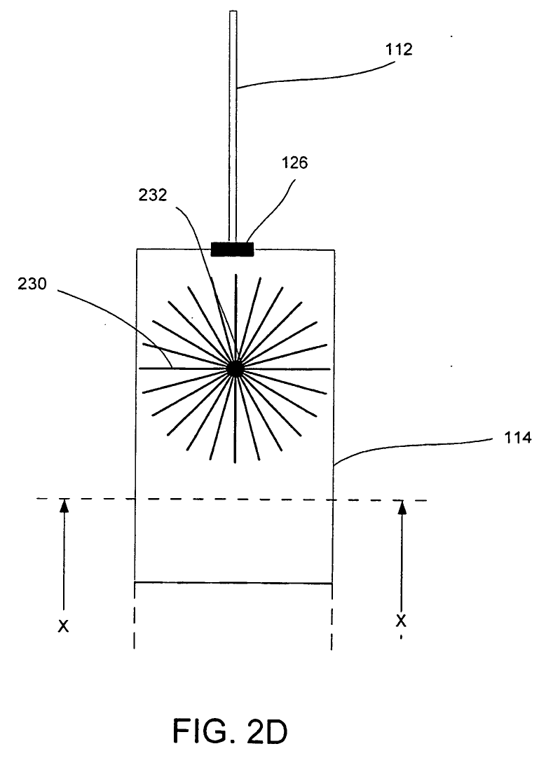

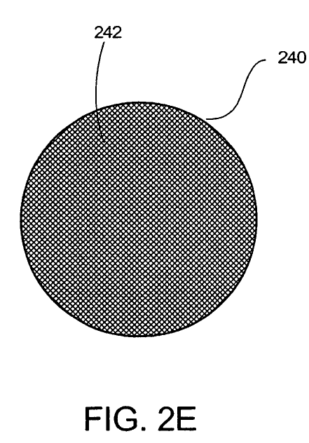

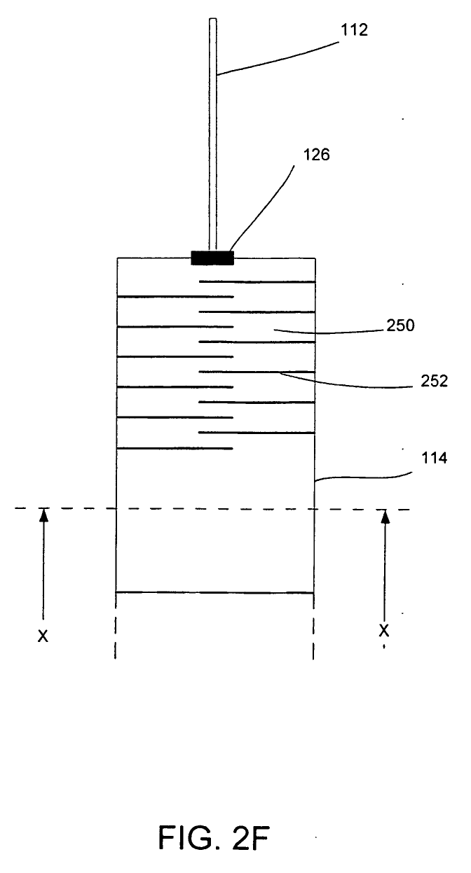

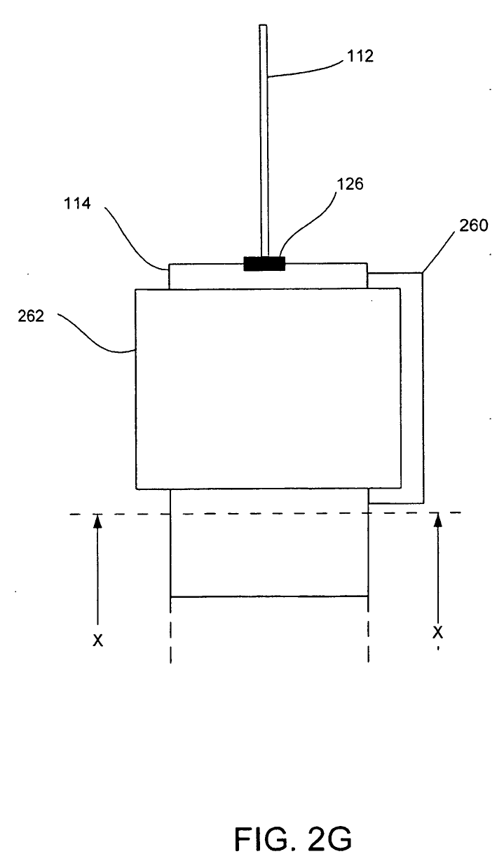

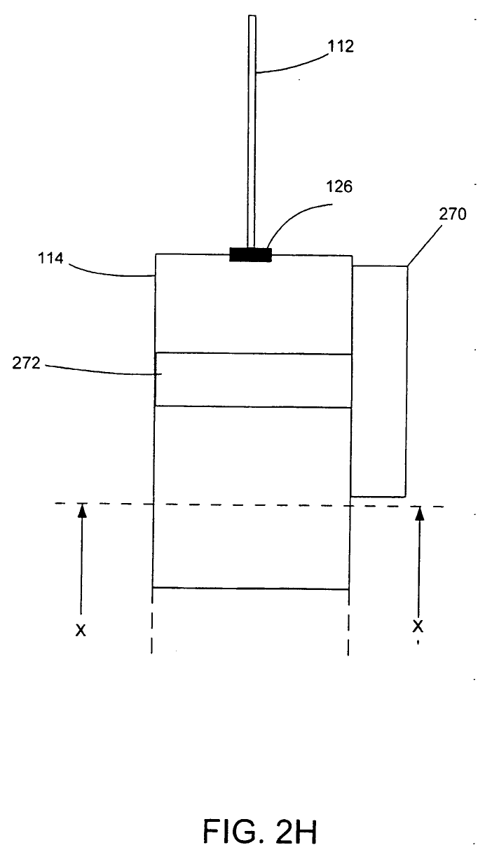

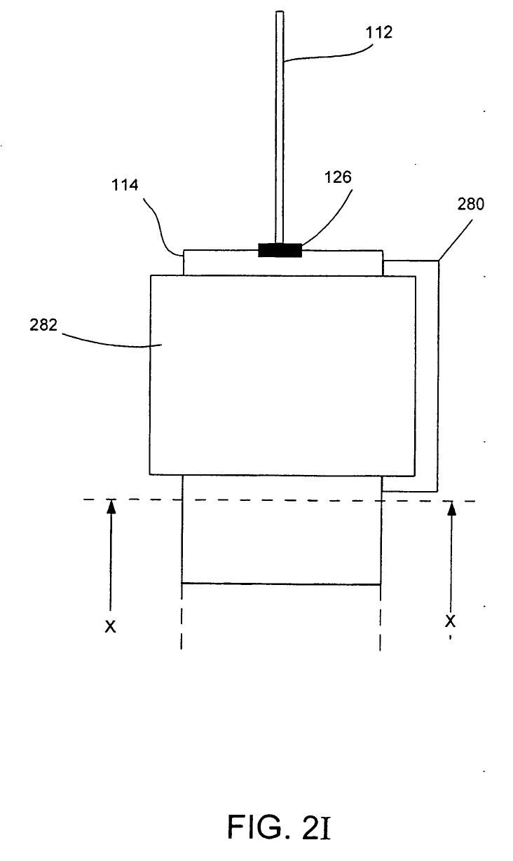

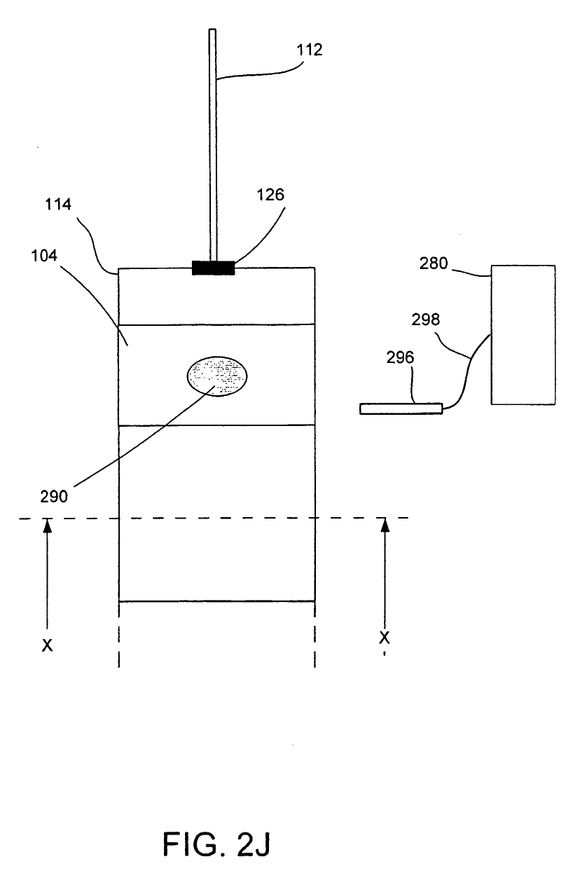

Once a tissue sample has been extracted from a patient into the chamber, the cells of the tissue sample are lysed using the lysis mechanism 104. Various embodiments of the lysis mechanism 104 are described below in relation to FIG. 2A-2J.

In a preferred embodiment, the treatment device 100 also includes an additive mechanism 98. In a specific embodiment, the additive mechanism 98 is generally similar to a typical syringe in that it includes an additive plunger 108 and an additive chamber 110. The additive mechanism 98 is fluidly connected to the chamber 114 at or near the lysis mechanism 104.

In a preferred embodiment, the additive mechanism 98 is coupled to the chamber 114 through a luer lock, permanent hose coupling, or the like. Also in a preferred embodiment, a one way valve 116 is provided to only allow fluid to flow from the additive chamber 110 to the chamber 114. In use, the additive chamber 110 preferably contains an additive solution 118, which increases the efficacy of the lysed tissue sample. In a preferred embodiment, the additive solution 118 can include one or more of the following, biological response modifiers for example, biological response modifiers, adjuvants, cytokines, antibodies, or agents such as anti-TGF beta antibody, anti-IL-10 antibody, soluble TGF-beta receptor, or soluble IL-10 receptor which counteract the immunosuppressive factors commonly present in tumor lysate. Additive solution 118 is added directly into the chamber 114 (FIG. 1) where the lysed tissue is located. The additives are preferably added through the one way valve 116 (FIG. 1) or through a self-sealing port 120 (FIG. 1), described below, in which the lysate sample is extracted for concentration/dosage determination and adjustment. Other additives, such as anti-cancer agents, immunostimulatory agents, anti-bacterial agents, anti-viral agents, or other drugs useful with the present invention, are described in further below.

Some adjuvants that may be added include, but are not limited to: saponin adjuvants, including without limitation QS-21, QS-7, GPI-100; heat shock proteins; alpha 2 macroglobulin; lipopolysaccharide (LPS); immunostimulatory oligonucleotides including CpG oligonucleotides; and complexes of heat shock proteins and antigenic molecules, such as peptides, or the like.

The following, United States patents by Srivastava, disclose heat shock proteins and complexes of heat shock proteins with antigenic molecules that can be added: U.S. Pat. Nos. 6,207,646, 6,194,388, 6,218,371, 6,239,116, 6,429,199, 6,406,705, 6,168,793; 6,048,530; 6,030,618; 6,017,540; 6,007,821; 5,997,873; 5,935,576; 5,837,251; and 5,750,119, all of which are incorporated herein by reference in their entireties.

According to an embodiment, the present invention can be used with one or more biological response modifiers which are immunostimulatory nucleic acids. Such nucleic acids, many of which are oligonucleotides comprising an unmethylated CpG motif, are mitogenic to vertebrate lymphocytes, and are known to enhance the immune response. See Woolridge, et al., 1997, Blood 89:2994-2998. Furthermore, the following patents and printed publications disclose immunostimulatory oligonucleotides which include CpG oligonucleotides that can be added: U.S. Pat. Nos. 6,207,646; 6,339,068; 6,239,116; 6,429,199; and PCT Patent publication, WO 01/22972, WO 00/06588, by Krieg et al.; WO 01/83503; WO 01/55370; and WO 01/12804 by Agrawal; WO 02/052002 by Fearon et al.; WO 01/35991 by Tuck et al.; WO 01/12223 by Van Nest; WO 98/55495; WO 99/62923 by Schwartz; U.S. Pat. No. 6,406,705 by Davis et al.; and PCT Patent publication WO 02/26757 by Kandimalla et al., all of which are incorporated herein by reference in their entireties.

Other kinds of immunostimulatory oligonucleotides such as phosphorothioate oligodeoxynucleotides containing YpG- and CpR-motifs have been described by Kandimalla et al. in “Effect of Chemical Modifications of Cytosine and Guanine in a CpG-Motif of Oligonucleotides: Structure-Immunostimulatory Activity Relationships.” Bioorganic & Medicinal Chemistry 9:807-813 (2001), incorporated herein by reference in its entirety. Also encompassed are immunostimulatory oligonucleotides that lack CpG dinucleotides which when administered by mucosal routes (including low dose administration) or at high doses through parenteral routes, augment antibody responses, often as much as did the CpG nucleic acids, however the response was Th2-biased (IgG1>>IgG2a). See United States Patent Publication No. 20010044416 A1, which is incorporated herein by reference in its entirety. Methods of determining the activity of immunostimulatory oligonucleotides can be performed as described in the aforementioned patents and publications. Moreover, immunostimulatory oligonucleotides can be modified within the phosphate backbone, sugar, nucleobase and internucleotide linkages in order to modulate the activity. Such modifications are known to those of skill in the art.

Furthermore, the following PCT Patent publications, by Srivastava, disclose alpha-2-macroglobulins that can be added: WO 01/91787, and WO 01/92474, both of which are incorporated herein by reference in their entireties.

Cytokines that preferably are added include but are not limited to: interleukin-1-alpha- (IL-1-alpha-), interleukin-1-beta- (IL-1-beta-), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interferon-alpha- (IFN-alpha-), interferon-beta- (IFN-beta-), interferon-gamma- (IFN-gamma-), tumor necrosis factor-alpha- (TNF-varies-), tumor necrosis factor-beta- (TNF-beta-), granulocyte colony stimulating factor (G-CSF), granulocyte/macrophage colony stimulating factor (GM-CSF), and transforming growth factor-beta- (TGF-beta-).

According to another embodiment, the invention may be used with complexes in combination with one or more biological response modifiers to treat cancer or infectious disease. One group of biological response modifiers is the cytokines. In one such embodiment, a cytokine is administered to a subject receiving HSP/α2M complexes. In another such embodiment, HSP/α2M complexes are administered to a subject receiving a chemotherapeutic agent in combination with a cytokine. In various embodiments, one or more cytokine(s) can be used and are selected from the group consisting of IL-1α, IL-1β, IL-2, IL-3, IL-4, IL-5, IL-6, IL-7, IL-8, IL-9, IL-10, IL-11, IL-12, IFNα, IFNβ, IFNγ, TNFα, TNFβ, G-CSF, GM-CSF, TGF-β, IL-15, IL-18, GM-CSF, INF-γ, INF-α, SLC, endothelial monocyte activating protein-2 (EMAP2), MIP-3α, MEP-3β, or an MHC gene, such as HLA-B7. Addtionally, other exemplary cytokines include other members of the TNF family, including but not limited to TNF-α-related apoptosis-inducing ligand (TRAIL), TNF-α-related activation-induced cytokine (TRANCE), TNF-α-related weak inducer of apoptosis (TWEAK), CD40 ligand (CD40L), lymphotoxin alpha (LT-α), lymphotoxin beta (LT-β), OX40 ligand (OX40L), Fas ligand (FasL), CD27 ligand (CD27L), CD30 ligand (CD30L), 41BB ligand (41BBL), APRIL, LIGHT, TL1, TNFSF16, TNFSF17, and AITR-L, or a functional portion thereof. See, e.g., Kwon et al., 1999, Curr. Opin. Immunol. 11:340-345 for a general review of the TNF family. Preferably, the HSP complexes or α2M complexes is administered prior to the treatment modalities. In a specific embodiment, complexes used with the present invention are administered to a subject receiving cyclophosphamide in combination with IL-12 for treatment of cancer.

In yet another embodiments, the device and method of the invention can be used with complexes in combination with one or more biological response modifiers which are agonists or antagonists of various ligands, receptors and signal transduction molecules of the immune system. For examples, the biological response modifiers include but are not limited to agoinsts of Toll-like receptors (TLR-2, TLR-7, TLR-8 and TLR-9; LPS; agonists of 41BB, OX40, ICOS, and CD40; and antagonists of Fas ligand, PD1, and CTLA-4. These agonists and antagonists can be antibodies, antibody fragments, peptides, peptidomimetic compounds, and polysaccharides.

Anti-immunosuppressive agents that may be added include but are not limited to: anti-4-1BB antibody, anti-TGF beta antibody, anti-IL-10 antibody, soluble TGF-beta receptor, and soluble IL-10 receptor.

Other suitable adjuvants, cytokines, and anti-immunosuppressive agents that can be added to chamber 114 to aid the lysed tissue sample's formation, administration, or efficacy can be found in A compendium of Vaccine Adjuvants and Excipients (2^{nd} Edition), Vogel, F., Powell, M., and Alving, C., in Vaccine Design—The Subunit and Adjuvant Approach, Powell, M., Newman, M., Burdman, J., Editors, Plenum Press, New York, 1995, pp. 141-227, and 2^{nd} Meeting on Novel Adjuvants Currently In/Close to Human Clinical Testing, World Health Organization—Organization Mondiale de la Sante Foundation Merieux, Annecy, France, 5-7 Jun. 2000, Kenney, R., Rabinovich, N. R., Pichyangkul, S., Price, V., and Engers, H., Vaccine, 20 (2002) 2155-63, all of which are incorporated herein by reference.

Some suitable antibodies that have in vivo therapeutic and/or prophylactic uses and may be added include, but are not limited to: MDX-010 (Medarex, N.J.) which is a humanized anti-CTLA-4 antibody; SYNAGIS® (MedImmune, Md.) which is a humanized anti-respiratory syncytial virus (RSV) monoclonal antibody for the treatment of patients with RSV infection; HERCEPTIN® (Trastuzumab) (Genentech, Calif.) which is a humanized anti-HER2 monoclonal antibody for the treatment of patients with metastatic breast cancer; REMICADE® (infliximab) (Centocor, Pa.) which is a chimeric anti-TNFα monoclonal antibody for the treatment of patients with Crone's disease; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection. Other examples are a humanized anti-CD18 F(ab′)₂ (Genentech); CDP860 which is a humanized anti-CD18 F(ab′)₂ (Celltech, UK); PRO542 which is an anti-HIV gp120 antibody fused with CD4 (Progenics/Genzyme Transgenics); Ostavir which is a human anti Hepatitis B virus antibody (Protein Design Lab/Novartis); PROTOVIR™ which is a humanized anti-CMV IgG1 antibody (Protein Design Lab/Novartis); MAK-195 (SEGARD) which is a murine anti-TNF-α F(ab′)₂ (Knoll Pharma/BASF); IC14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DIAGNOSTIC REAGENT antibody (Techniclone); ABX-IL8 is a human anti-IL8 antibody (Abgenix); anti-CD11a is a humanized IgG1 antibody (Genentech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); SCH55700 is a humanized anti-IL-5 IgG4 antibody (Celltech/Schering); SB-240563 and SB-240683 are humanized anti-IL-5 and IL-4 antibodies, respectively, (SmithKline Beecham); rhuMab-E25 is a humanized anti-IgE IgG1 antibody (Genentech/Norvartis/Tanox Biosystems); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β₂-integrin IgG antibody (LeukoSite); Anti-LFA-1 is a murine anti CD18 F(ab′)₂ (Pasteur-Merieux/Immunotech); CAT-152 is a human anti-TGF-02 antibody (Cambridge Ab Tech); and Corsevin M is a chimeric anti-Factor VII antibody (Centocor) and anti-4-1BB antibody. The above-listed immunoreactive reagents, as well as any other immunoreactive reagents, may be administered according to any regimen known to those of skill in the art, including the regimens recommended by the suppliers of the immunoreactive reagents. In a preferred embodiment an anti-CTLA4 or and anti-4-IBB antibody are used with the device and method of the present invention. More additives useful in conjunction with the present invention are described below.

In another embodiment, a self-sealing port 120 may be provided in the treatment device 100. The self-sealing port 120 is preferably located in a wall of the chamber 114 at or near the lysis mechanism 104. The self-sealing port is preferably composed of a resilient medical grade rubber substance that can be pierced by a needle and which self-seals when the needle is withdrawn. In use, an additive solution may be injected into the chamber 114 via the self-sealing port 120, or a sample of the lysed tissue sample may be withdrawn through the self-sealing port for quantitation, or the like.

The treatment device 100 of FIG. 1 also consists of an administration mechanism that is preferably similar to a typical syringe for administering hypodermic or subcutaneous injections. In this embodiment, the administration mechanism includes an administration needle 124 coupled to the syringe type device 102 (FIG. 1 shows the administration needle 124 uncoupled to the syringe type device 102). The administration needle 124 is in fluid communication with the lysed tissue sample within the chamber 114. In use, once a lysed tissue sample has been produced within the chamber 114, the collection needle 112 is replaced with the administration needle 124. The administration needle 124 is preferably either a standard hypodermic injection needle, a standard subcutaneous injection needle, a standard scarify administration device, or the like. The administration needle 124 generally has a smaller diameter than the collection needle 112.

FIG. 2A is a diagrammatic plan view of a lysis mechanism 104 for causing controlled physical lysis of a tissue or tumor sample, according to an embodiment of the invention. Lysis mechanism 104 is contained substantially within the chamber 114 of the treatment device 100 of FIG. 1. In all of the following embodiments of the lysis mechanism 104, it should be appreciable to those skilled in the art that the overall dimensions of the lysis mechanism 104 are preferably restricted to encompass a small overall surface area. In other words, the more surface area exposed to the lysed tissue sample within the lysis mechanism, the more proteins will adhere to the surface area, causing the dosage to be diluted and not be available for administration into the patient. A suitable surface area approximates that of, or would work in conjunction with, a 3 cubic centimeter (cm³) chamber 114. Preferably, chamber 114 is in the range of 0.1 to 3 cm³; and in a specific embodiment is 0.5 cm³. Furthermore, to help minimize any adhesion of proteins to the surfaces of the lysis mechanism 104, all surfaces that potentially come into contact with the lysed tissue are preferably coated with a substance that inhibits protein adhesion; thus, for example, the surfaces can be either siliconized surfaces or constructed from or coated with Teflon® made by DuPont or Dalkyo Resin CZ® made by Dalkyo Seiko Ltd. of Japan, or the like.

In the embodiment of FIG. 2A, lysis mechanism 104 is composed of cylinders 200A and 200B that are aligned adjacent to one another, such that their longitudinal axes are substantially parallel. The cylinders 200A and 200B are preferably sealed within the chamber 114 and are rotatably supported on axles 202A and 202B, respectively, that are coupled to the wall of the chamber 114. Although the chamber 114 preferably has a circular cross section elsewhere, it preferably has a rectangular cross section around the cylinders 200A and 200B, as shown in FIG. 2B below. Also, the cylinders 200A and 200B are preferably rotatable about 360 degrees as depicted by arrows 206A and 206B.

FIG. 2B is a cross sectional view of the lysis mechanism shown in FIG. 2A, as viewed along line X-X′ of FIG. 2A. The cylinders 200A and 200B are spaced a predetermined distance from one another to cause controlled physical lysis of the tissue sample passing between the cylinders when they are rotated. Also, the surface of cylinders 200A and 200B may include a surface texture 212A and 212B to optimize lysis of the sample tissue passing between the cylinders 200A and 200B. In addition, the surfaces of cylinders 200A and 200B are preferably constructed out of, or coated with, a non-stick substance, such as TEFLON® made by DuPont, Dalkyo Resin CZ® made by Dalkyo Seiko Ltd. of Japan, or siliconized surfaces so that the lysed tumor tissue cells will not substantially adhere to either of the cylinders 200A or 200B.

In use, when a pressure differential is created on both sides of the cylinders 200A and 200B (FIG. 2A), such as when suction is created by retracting plunger 106 (FIG. 1) away from cylinders 200A and 200B, the tissue sample is lysed as it passes between the rotating cylinders 200A and 200B.

In another embodiment, at least one end of at least one axle 202A or 202B protrudes outside the chamber 114 through a seal 201. A rotation mechanism may then be either permanently or removably coupled to one or both axles 202A and 202B, whichever protrudes from the chamber 114. When rotated, at a predetermined speed, the rotation mechanism rotates the cylinders to cause adequate cell lysis. Suitable rotation mechanisms include one or more motors, a hand crank 150 (FIG. 1), a high speed rotating mechanism 152 (FIG. 1), or the like. An example of a suitable high speed rotating mechanism 152 (FIG. 1) is the Brinkmann/KINEMATICA POLYTRON Handheld Homogenizer Model PT 1200C or 1300D made by Brinkmann Instruments Inc., Westbury, N.Y.

FIG. 2C is a diagrammatic plan view of another embodiment of the lysis mechanism 104 shown in FIG. 1, according to another embodiment of the invention. In this embodiment, the lysis mechanism comprises two intermeshing rotatable gears 220A and 220B, which are similar to the cylinders 200A and 200B of FIG. 2A. The tolerance 208 between the gears 220A and 220B is chosen to cause controlled physical lysis of a particular tissue sample when the tissue sample passes between the gears 220A and 220B.

FIG. 2D is a diagrammatic plan view of yet another embodiment of the lysis mechanism 104 shown in FIG. 1, according to yet another embodiment of the invention. This lysis mechanism 104 includes one or more blades 230, otherwise known as a mixer or blender. A suitable example of blade(s) 230 is the Brinkmann/KINEMATIC POLYTRON® Generators made by Brinkmann Instruments Inc., Westbury, N.Y. In use, an axle 232 coupled to the blades is rotated to cause lysis of the tissue sample. In a preferred embodiment, one end of the axle 232 protrudes from chamber 114 through a seal. The protruding end of the axle 232 is configured to adapt to a rotating mechanism, as described above. For example, in one embodiment, rotating the blades 230 at 5000 RPM for approximately 15 seconds causes sufficient lysis. Furthermore, the blades 230 may be coated with a non-stick surface such as TEFLON® made by DuPont, Dalkyo Resin CZ® made by Dalkyo Seiko Ltd. of Japan, siliconized surfaces, or the like, so the lysed cellular tissue does not stick to the blades 230.

FIG. 2E is a cross sectional view of even another lysis mechanism, as viewed along line X-X′ of FIG. 1. In this embodiment, the lysis mechanism 104 comprises a grate 240 that is used to lyse the tissue sample. The grate 240 includes a plurality of the holes 242 having a predetermined diameter to cause controlled physical lysis of a particular tissue sample when the cells are forced to pass through the grate 240. In use, when a tissue sample is drawn through the grate 240 by suction generated by moving the plunger 106 away from, or towards, the grate 240, the tissue sample is forced through the holes 240, thereby causing lysis of the tissue sample.

FIG. 2F is a diagrammatic plan view of yet another embodiment of the lysis mechanism 104 shown in FIG. 1. In this embodiment, the lysis mechanism comprises a tortuous path 250 made from staggered or interdigitating walls 252. In use, as the plunger 106 is moved away from and/or towards the tortuous path 250, the tissue sample is forced through the tortuous path, thereby causing lysis of the tissue cells.

FIG. 2G is a diagrammatic plan view of yet another alternative embodiment of the lysis mechanism. In this embodiment, the lysis mechanism 104 comprises a cooling mechanism 260 and a cooling jacket 262. The cooling jacket 262 surrounds the chamber 114. A series of cooling and warming fluids are introduced into the cooling jacket 262, causing the tissue to undergo alternate cooling and warming cycles which cause controlled physical cell lysis. An example of a suitable cooling and warming cycle is subjecting the tissue sample to liquid nitrogen for between 5 seconds to 10 minutes and then to water at between 32-42 degrees Celsius, and preferably 37 degrees Celsius for between 5 seconds to 10 minutes.

FIG. 2H is a diagrammatic plan view of still another alternative embodiment of the lysis mechanism. In this embodiment, the lysis mechanism comprises a cooling mechanism 270 and a heat exchanger 272 contained within the chamber 114. In use, after the tissue sample is collected it is subjected to a series of cooling and warming cycles. The cooling and warming produced by the cooling mechanism 270 and heat exchanger 272 cause controlled physical lysis of the collected tissue sample.

FIG. 2I is a diagrammatic plan view of still another alternative embodiment of the lysis mechanism that causes cell lysis by sonication. In this embodiment, the lysis mechanism comprises an ultrasonic mechanism 280 and an ultrasonic jacket 282. In use, the ultrasonic mechanism 280 produces an ultrasonic pressure wave that, through the ultrasonic jacket 282, subjects the collected tissue sample to ultrasonic forces, that in turn causes controlled physical lysis of the collected tissue sample.

FIG. 2J is a diagrammatic plan view an alternative embodiment of the lysis mechanism that causes cell lysis by sonication. In this embodiment, the lysis mechanism comprises an ultrasonic probe 296 connected to an ultrasonic mechanism 280 by conducting wire(s) 298. In use, the ultrasonic probe 296 is inserted into the chamber 114 through a port 290. Port 290 is similar to the self-sealing port 120 previously described. When the ultrasonic probe 296 comes in contact with, or comes near to, the tissue sample contained within the chamber 114, a ultrasonic pressure wave generated by the ultrasonic probe 296 causes controlled physical lysis. Alternatively, the ultrasonic probe 296 can be permanently mounted within the chamber 114.

Although only a few embodiments of the lysis mechanism 104 are described above, it should be appreciated that any suitable lysis mechanism 104 may be employed to cause lysis of the cells of the tissue sample.

### 5.2 Methods for Generating Lysed Tissue Samples

The present invention includes a method of creating a lysed tissue sample for the treatment of disease and/or cancer or stimulation of the immune system (e.g., induction or enhancement of an immune response). The lysed tissue sample is generated by utilizing the patients own diseased and/or cancerous tissue, lysing and homogenizing the tissue, treating the lysed tissue with an additive solution, and administering the lysed tissue sample to the patient.

FIG. 3 is a flow chart of method 300 of producing and administering a lysed tissue sample for a disease or cancer by using the self contained treatment device of the present invention. The method utilizes the treatment device 100 described above. A collection needle 112 (FIG. 1) is initially attached to a chamber 114 (FIG. 1) of a treatment device 100 (FIG. 1), at step 302. The collection needle is then inserted into the diseased area of the patient or into surgically removed tissue from a patient or another patient, at step 304. In a preferred embodiment the collection needle is inserted into a tumor's core.

A tissue sample is then extracted into the chamber 114 (FIG. 1), at step 306. In a preferred embodiment the tissue sample is tissue from the tumor's core. Also in a preferred embodiment, the tissue sample is extracted by retracting or withdrawing the plunger 106 (FIG. 1), as described above. It should be appreciated that any suitable extraction device may be used to extract the tissue sample. Also in a preferred embodiment, chamber 114 (FIG. 1), contains a solution, preferably sterile, to facilitate lysis of the tissue sample and administration of the lysed tissue sample. Suitable solutions include, but are not limited to, a saline solution, a saline solution containing a surfactant such as Tween® 80 (polyoxyethylene sorbitan monooleate) or Tween® 20 (polyoxyethylene sorbitan monolaurate) made by Huanan Chemical and Industrial Corp., China or a saline solution containing sugars such as glycerol or polyethylene glycol (PEG). Such solutions would ideally facilitate lysis of the tissue sample, minimize adsorption of proteins to the surfaces of the treatment device, and facilitate administration of the lysed tissue sample.

A lysed tissue sample is then produced by lysing the tissue sample within the chamber 114 (FIG. 1), at step 308. Lysis may occur using any of the lysis mechanisms or techniques described above in relation to FIGS. 2A-2J. It should also be appreciated that any suitable lysis mechanisms may be used to lyse the tissue sample. Also, it is preferred that the lysis mechanism does not denature proteins, and thus, preferably does not subject the tissue sample to denaturing conditions known in the art.

The lysis mechanism may physically squash, grind, blend, or grate the tissue sample, such as by passing the tissue sample through the cylinders, gears, or grate described above. Lysis is controllable by controlling the clearance or tolerance between the gears or cylinders, the grating or tortuous path sizes, the speed of rotation, the rotation time, or the like.

Another embodiment consists of treating the collected sample to repetitive cooling and warming cycles, such that the cellular membranes are caused to undergo lysis. A preferable cooling and warming cycle includes cooling the tissue sample for between 5 seconds to 10 minutes with liquid nitrogen via the cooling jacket 262, and then subjecting the sample to water at between 32-42 degrees Celsius and preferably 37 Celsius for between 5 seconds to 10 minutes via the cooling jacket 262 surrounding the chamber 114. Repeating these steps causes lysis of the cellular matter. This technique is described above in relation to FIG. 2G and FIG. 2H.

A further embodiment for lysis is to treat the sample by sonication to break open the tissue or tumor cells, as described above in relation to FIGS. 2I and 2J. In one configuration of this embodiment, an ultrasonic jacket 282 (FIG. 2I) delivers a ultrasonic force to the tissue sample causing lysis. In another configuration, an ultrasonic probe 296 is either inserted into the chamber 114 through a port 290 to deliver a ultrasonic force to the tissue sample or the ultrasonic probe 296 is permanently embedded within the chamber 114.

Furthermore, as an optional step, to monitor the degree of lysis achieved a small sample volume of the lysate may be retrieved through the self-sealing port 120 (FIG. 1) on the treatment device, at step 310, and examined microscopically. Alternatively, for example, the protein concentration of the lysate can be determined by traditional protein assay techniques such as the Bradford assay, ultraviolet based techniques, or the like. Such a step, 310, is used to monitor quality control, determine and develop highly accurate dosages, set an optimal dosage for any particular treatment, or the like. However, generally the protein concentration is determined by calculating standard cell equivalents from the known volume of tissue sample retrieved.

To determine cell equivalents, the volume of the extracted tissue sample is determined after extracting the tissue. In one embodiment of the invention, the volume of extracted tissue can be determined by comparing the extracted tissue contained within the chamber 114 with the graduated unit volume markings 115 (FIG. 1) on the wall of the chamber 114. A dose of lysed tissue sample that can be administered to the patient is then determined based on cell equivalents. Cell equivalents are the approximation of the number of cells that constitute a given volume of tissue. For example, it has been determined that one cubic centimeter (1000 cubic millimeters) of tissue contains approximately 1×10⁸-1×10⁹ cells. Thus, following the extraction of one cubic centimeter of tissue from a patient, lysis of the tissue, and administration of the entire lysed tissue sample into the patient, the patient will have been administered a dosage of approximately 1×10⁸-1×10⁹ cell equivalents. The following table indicates the amounts of tissue, expressed in volume (mm³), that may be extracted from a patient and the approximate cell equivalents (number of cells) associated with that given volume:

| | |
|---|---|
| Volume of Tissue | Cell Equivalents |
| (mm³) | (number of cells present) |

| | | |
|---|---|---|
| 0.01 | mm³ | 1 × 10³-1 × 10⁴ |
| .1 | mm³ | 1 × 10⁴-1 × 10⁵ |
| 1 | mm³ | 1 × 10⁵-1 × 10⁶ |
| 10 | mm³ | 1 × 10⁶-1 × 10⁷ |
| 100 | mm³ | 1 × 10⁷-1 × 10⁸ |
| 1000 | mm³ | 1 × 10⁸-1 × 10⁹ |

However, the entire volume of extracted tissue need not be administered to the patient. A proportion of the lysed tissue sample, such as for example, 1/10th, ⅕th, ¼th, ½ or ¾ of the extracted tissue volume can be administered. Furthermore, the remaining portion can be retained for later treatment administrations if desired.

In another embodiment, an additive solution may then be added to the lysed tissue sample, at step 312, using the additive mechanism(s) described above. For example, adjuvants, cytokines, antibodies, and agents such as anti-TGF beta antibody, anti-IL-10 antibody, soluble TGF-beta receptor, soluble IL-10 receptor which counteract the immunosuppressive factors present in tumor lysate, as described above, are added to the lysed tissue. These additives are added directly into the chamber 114 (FIG. 1) where the lysed tissue is located. The additives are preferably added through the one way valve 116 (FIG. 1) or through a self-sealing port 120 (FIG. 1) through which the lysate sample was extracted for concentration/dosage determination and adjustment.

According to another embodiment, both the lysed tissue sample and any additive, only the lysed tissue sample, only one additive, multiple additives, or any combination of these with or without a saline, buffer, dilutant, or the like, can be added to the chamber 114 prior to administration. Furthermore, the patient may be the source of the tissue sample, another mammal may be the source of the tissue sample, the tissue sample may be derived from cell culture, or the like. It is further contemplated that the tissue sample may be added to the chamber 114 with or without any additive prior to administration to the patient.

Because saline solution and other additives may be added to the lysed tissue sample prior to administration, as described above, the overall volume of the lysed tissue sample may include the volume of the additives and/or saline solution. These additional volumes should be factored into a dosage determination. For example, purely by way of explanation and not limitation, consider one cubic centimeter of tissue containing approximately 1×10⁸ cells extracted and lysed in the presence of 2 milliliters of saline solution. The total volume of the mixture, including the lysed tissue sample and the saline solution, may then equal approximately 3 milliliters but the cell equivalents remain at approximately 1×10⁸ cells. If it is desired to then administer 1×10⁷ cell equivalents to a patient, approximately 1/10th of the total volume of material in the treatment device is then administrated to the patient. Accordingly, dosages of lysed tissue sample range from 1×10³ cell equivalents to 1×10⁹ cell equivalents, and preferably from about 1×10⁵-1×10⁷ cell equivalents.

According to an embodiment, the device and method of the invention is used with complexes in combination with one or more adjuvants Some adjuvants that may be added include but are not limited to: saponin adjuvants, including without limitation, QS-21, QS-7, and GPI-100, heat shock proteins, complexes of heat shock proteins and antigenic molecules, alpha 2 macroglobulin, lipopolysaccharide (LPS), alum (e.g., aluminum hydroxide, aluminum phosphate), emulsion based formulations (e.g., Montanide and MF-59,) lipid A derivatives, (e.g., monophosphoryl lipid A (MPL)), aminoalkyl glucosaminide phosphates, ISCOMs, bacterial toxins (e.g., cholera toxin (CT), E. Coli heat labile enterotoxin, labile toxin (LT), pertussis toxin (PT) and derivatives thereof), or the class of adjuvants known as “immunostimulatory nucleic acids or immunostimulatory oligonucleotides” which includes “CpG oligonucleotides”. Other suitable adjuvants and additives include cytokines, antibodies, and anti-immunosuppressive agents such as anti-TGF beta antibody, anti-IL-10 antibody, soluble TGF-beta receptor, soluble IL-10 receptor and those previously listed and incorporated herein by reference. According to an embodiment, the device and method of the invention is used with complexes in combination with one or more adjuvants. The adjuvant(s) can be administered separately or present in a composition. A systemic adjuvant is an adjuvant that can be delivered parenterally. Systemic adjuvants include adjuvants that creates a depot effect, adjuvants that stimulate the immune system and adjuvants that do both. An adjuvant that creates a depot effect as used herein is an adjuvant that causes the antigen to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

Other adjuvants stimulate the immune system, for instance, cause an immune cell to produce and secrete cytokines or IgG. This class of adjuvants includes but is not limited to immunostimulatory nucleic acids, such as CpG oligonucleotides; saponins purified from the bark of the Q. saponaria tree, such as QS21; poly[di(carboxylatophen-oxy)phosphazene (PCPP polymer; Virus Research Institute, USA); derivatives of lipopolysaccharides (LPS) such as monophosphoryl lipid A (MPL; Ribi ImmunoChem Research, Inc., Hamilton, Mont.), muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Other systemic adjuvants are adjuvants that create a depot effect and stimulate the immune system. These compounds are those compounds which have both of the above-identified functions of systemic adjuvants. This class of adjuvants includes but is not limited to ISCOMs (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

Yet other systemic adjuvants can include, by way of example and not limitation bacterial toxins, such as Cholera toxin (CT), Excherichi coli heat-liable enterotoxin, Labile toxin (LT), Pertussis toxin (PT) and derivatives thereof, and Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983).

The following, U.S. patents by Srivastava, disclose heat shock proteins and complexes of heat shock proteins with antigenic molecules that can be added to the lysed tissue sample: U.S. Pat. Nos. 6,168,793; 6,048,530; 6,030,618; 6,017,540; 6,007,821; 5,997,873; 5,935,576; 5,837,251; and 5,750,119, all of the forgoing are incorporated herein by reference in their entireties.

Furthermore, the following patents and printed publications disclose adjuvants known as immunostimulatory oligonucleotides which include CpG oligonucleotides that can be added: U.S. Pat. Nos. 6,207,646; 6,339,068; 6,239,116; 6,429,199; and PCT Patent publication, WO 01/22972, WO 00/06588, by Krieg et al.; WO 01/83503; WO 01/55370; and WO 01/12804 by Agrawal; WO 02/052002 by Fearon et al.; WO 01/35991 by Tuck et al.; WO 01/12223 by Van Nest; WO 98/55495; WO 99/62923 by Schwartz; U.S. Pat. No. 6,406,705 by Davis et al.; and PCT Patent publication WO 02/26757 by Kandimalla et al., all of the forgoing are incorporated herein by reference in their entireties.

Furthermore, the following PCT Patent publications, by Srivastava, disclose alpha-2-macroglobulins that can be added: WO 01/91787, and WO 01/92474, both of which are incorporated herein by reference in their entireties.

In a preferred embodiment, the collection needle 112 (FIG. 1) is then replaced with the administration needle 124 (FIG. 1), at step 314. Alternatively, the same needle may be used for both extraction and administration. The administration needle is then inserted into the patient, at step 316. In a preferred embodiment, the administration needle is inserted at a different location than the tumor, to avoid the immunosuppressive environment created by some tumors. Finally, the lysed tissue sample is administered into the patient, at step 318. In a preferred embodiment, administration occurs by depressing the plunger 106 (FIG. 1) of the treatment device to expel the lysed tissue sample. Suitable examples of appropriate routes of administration include, but are not limited to: intradermally, intravenously, subcutaneously, intramuscularly, intra-orbitally, ophthalmically, intraventricularly, intracranially, intracapsularly, intraspinally, intracisternally, intraperitoneally, intrabuccally, intrarectally, intravaginally, or the like.

All or a portion of the treatment device's lysed tissue sample contents can be administered, depending on the desired dosage. By way of explanation but not limitation, following administration, the host's immune system recognizes the immunogenic components of the lysate including the heat shock protein-peptide complexes. An immune response is then generated that is able to attack diseased cells expressing the peptides in the lysed tissue sample.

Compositions, which comprise complexes of antigenic peptides derived from digested cytosolic and/or membrane-derived proteins of antigenic cells or viral particle and a HSP and/or α2M, is administered to The device and methods of the present invention are useful for the prevention and treatment of a subject with cancer or/and infectious diseases in accordance with the device and methods of the present invention. In one embodiment, “treatment” or “treating” refers to an amelioration of cancer or an infectious disease, or at least one discernible symptom thereof. In another embodiment, “treatment” or “treating” refers to an amelioration of at least one measurable physical parameter associated with cancer or an infectious disease, not necessarily discernible by the subject. In yet another embodiment, “treatment” or “treating” refers to inhibiting the progression of a cancer or an infectious disease, either physically, e.g., stabilization of a discernible symptom, physiologically, e.g., stabilization of a physical parameter, or both.

In certain embodiments, the device and methods of the present invention are used to develop and/or administer compositions to a subject as a preventative measure against such cancer or an infectious disease. As used herein, “prevention” or “preventing” refers to a reduction of the risk of acquiring a given cancer or infectious disease. In one mode of the embodiment, the device and methods of the present invention administer a preventative measure to a subject having a genetic predisposition to a cancer. In another mode of the embodiment, the device and methods of the present invention administer a preventive measure to a subject facing exposure to carcinogens including but not limited to chemicals and/or radiation, or to a subject facing exposure to an agent of an infectious disease.

As used throughout this application, a combination therapy refers to the use of HSP complexes or α2M complexes the device of the present invention with another modality to prevent or treat cancer and infectious diseases. The administration of the complexes with the device and methods of the present invention can augment the effect of anti-cancer agents or anti-infectives, and vice versa. Preferably, this additional form of modality is a non-HSP and non-α2M based modality, i.e., this modality does not comprise either HSP or α2M as a component. This approach is commonly termed combination therapy, adjunctive therapy or conjunctive therapy (the terms are used interchangeably herein). With combination therapy, additive potency or additive therapeutic effect can be observed. Synergistic outcomes where the therapeutic efficacy is greater than additive can also be expected. The use of combination therapy can also provide better therapeutic profiles than the administration of the treatment modality, or the HSP complexes or α2M complexes use of the device and methods of the invention alone. The additive or synergistic effect may allow the dosage and/or dosing frequency of either or both modalities be adjusted to reduce or avoid unwanted or adverse effects.

### 5.3 Various Treatment Regimes Utilizing the Invention

In one embodiment, combination therapy encompasses the adjunctive uses of one or more modalities that aid in the prevention or treatment of cancer, which modalities include, but are not limited to chemotherapeutic agents, immunotherapeutics, anti angiogenic agents, cytokines, hormones, antibodies, polynucleotides, radiation and photodynamic therapeutic agents. In specific embodiments, combination therapy can be used to prevent the recurrence of cancer, inhibit metastasis, or inhibit the growth and/or spread of cancer or metastasis.

Some types of cancers that can be treated or prevented by the device and methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease.

An anti-cancer agent can be chemotherapeutic agents, which include but are not limited to, the following groups of compounds: cytotoxic antibiotics, antimetabolities, anti-mitotic agents, alkylating agents, platinum compounds, arsenic compounds, DNA topoisomerase inhibitors, taxanes, nucleoside analogues, plant alkaloids, and toxins; and synthetic derivatives thereof. Table 1 lists exemplary compounds of the groups:

TABLE 1

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| Platinum containing compounds: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxol |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| mitomycins: | Mitomycin C |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | |
| | Deferoxamine |
| Pyrimidine analogs: | |
| Uracil analogs: | 5-Fluorouracil |
| | Floxuridine |
| | Doxifluridine |
| | Ratitrexed |
| Cytosine analogs: | Cytarabine (ara C) |
| | Cytosine arabinoside |
| | Fludarabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | pyrazoloimidazole |
| Antimitotic agents: | allocolchicine |
| | Halichondrin B |
| | colchicine |
| | colchicine derivative |
| | dolstatin 10 |
| | maytansine |
| | rhizoxin |
| | thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ATPase inhibitors: | Thapsigargin |

In a preferred embodiment, the chemotherapeutic agent is one or more of the following: gemcitabine, irinotecan, fluorouracil (e.g. 5-fluorouracil), capecitabine, topotecan, vinorelbine, docetaxel, paclitaxel, reltitrexed, daunorubicin, doxorubicin, oxaliplatin, cisplatin.

In another embodiment, the device and method generates and/or administers complexes in combination with one or more immunotherapeutic agents, such as antibodies and vaccines. In a preferred embodiment, the antibodies have in vivo therapeutic and/or prophylactic uses against cancer. In some embodiments, the antibodies can be used for treatment and/or prevention of infectious disease. Examples of therapeutic and prophylactic antibodies include, but are not limited to, MDX-0110 (Medarex, N.J.) which is a humanized anti-CTLA-4 antibody currently in clinic for the treatment of prostate cancer; SYNAGIS® (MedImmune, Md.) which is a humanized anti-respiratory syncytial virus (RSV) monoclonal antibody for the treatment of patients with RSV infection; HERCEPTIN® (Trastuzumab) (Genentech, Calif.) which is a humanized anti-HER2 monoclonal antibody for the treatment of patients with metastatic breast cancer. Other examples are a humanized anti-CD18 F(ab′)2 (Genentech); CDP860 which is a humanized anti-CD 18 F(ab′)2 (Celltech, UK); PRO542 which is an anti-HIV gp120 antibody fused with CD4 (Progenics/Genzyme Transgenics); Ostavir which is a human anti Hepatitis B virus antibody (Protein Design Lab/Novartis); PROTOVIR™ which is a humanized anti-CMV IgG1 antibody (Protein Design Lab/Novartis); MAK-195 (SEGARD) which is a murine anti-TNF-α F(ab′)2 (Knoll Pharma/BASF); IC14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DIAGNOSTIC REAGENT antibody (Techniclone); ABX-IL8 is a human anti-IL8 antibody (Abgenix); anti-CD11a is a humanized IgG1 antibody (Genentech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); SCH55700 is a humanized anti-IL-5 IgG4 antibody (Celltech/Schering); SB-240563 and SB-240683 are humanized anti-IL-5 and IL-4 antibodies, respectively, (SmithKline Beecham); rhuMab-E25 is a humanized anti-IgE IgG1 antibody (Genentech/Norvartis/Tanox Biosystems); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β2-integrin IgG antibody (LeukoSite); Anti-LFA-1 is a murine anti CD18 F(ab′)2 (Pasteur-Merieux/Immunotech); CAT-152 is a human anti-TGF-β2 antibody (Cambridge Ab Tech); and Corsevin M is a chimeric anti-Factor VII antibody (Centocor). The above-listed immunoreactive reagents, as well as any other immunoreactive reagents, may be administered according to any regimen known to those of skill in the art, including the regimens recommended by the suppliers of the immunoreactive reagents.

In another embodiment a vaccine, the device and method of the present invention generates and/or administers complexes in combination with one or more anti angiogenic agents, which includes, but is used in combination with the present invention. Suitable vaccines include live or attenuated vaccines as well as subunit and synthetic vaccines. Many such vaccines are known in the art and are in development for cancer and infectious diseases; examples of such vaccines for humans are described in The Jordan Report 2000, Accelerated Development of Vaccines, National Institute of Health, which is incorporated herein by reference in its entirety. In a preferred embodiment, the invention is used in combination with an heat shock protein or alpha 2 macroglobulin based vaccine as described in U.S. Pat. Nos. 5,837,251; 6,410,027; 6,017,540; 5,961,979; 6,455,503; 5,935,576 and international patent No.'s EP700445; WO 99/22761; WO 97/06821; WO 01/91787; and in Hoos and Levey (2003) Expert Rev. Vaccines 2(3):369-79; Manjili et al. (2002) Frontiers Bioscience 7:d43-52, all of which are incorporated herein by reference in their entirity.

In another embodiment, the device and method of the present invention are used in combination with one or more anti angiogenic agents, which includes, but are not limited to, angiostatin, thalidomide, kringle 5, endostatin, Serpin (Serine Protease Inhibitor) anti thrombin, 29 kDa N-terminal and a 40 kDa C terminal proteolytic fragments of fibronectin, 16 kDa proteolytic fragment of prolactin, 7.8 kDa proteolytic fragment of platelet factor 4, a 13 amino acid peptide corresponding to a fragment of platelet factor 4 (Maione et al., 1990, Cancer Res. 51:2077 2083), a 14 amino acid peptide corresponding to a fragment of collagen I (Tolma et al., 1993, J. Cell Biol. 122:497 511), a 19 amino acid peptide corresponding to a fragment of Thrombospondin I (Tolsma et al., 1993, J. Cell Biol. 122:497 511), a 20 amino acid peptide corresponding to a fragment of SPARC (Sage et al., 1995, J. Cell. Biochem. 57:1329 1334), or any fragments, family members, or variants thereof, including pharmaceutically acceptable salts thereof.

Other peptides that inhibit angiogenesis and correspond to fragments of laminin, fibronectin, procollagen, and EGF have also been described (see, e.g., Cao, 1998, Prog Mol Subcell Biol. 20:161 176). Monoclonal antibodies and cyclic pentapeptides, which block certain integrins that bind RGD proteins (i.e., possess the peptide motif Arg Gly Asp), have been demonstrated to have anti vascularization activities (Brooks et al., 1994, Science 264:569 571; Hammes et al., 1996, Nature Medicine 2:529 533). Moreover, inhibition of the urokinase plasminogen activator receptor by receptor antagonists inhibits angiogenesis, tumor growth and metastasis (Min et al., 1996, Cancer Res. 56: 2428 33; Crowley et al., 1993, Proc Natl Acad. Sci. 90:5021 25). Use of such anti angiogenic agents in combination with the complexes is also contemplated by the present invention.

In yet another embodiment, the device and methods of the present invention are used in association with a hormonal treatment. Hormonal therapeutic treatments comprise hormonal agonists, hormonal antagonists (e.g., flutamide, bicalutamide, tamoxifen, raloxifene, leuprolide acetate (LUPRON), LH RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), and antiandrogens (e.g., cyproterone acetate).

In yet another embodiment, the device and methods of the present invention are used in association with a gene therapy program in the treatment of cancer. In one embodiment, gene therapy with recombinant cells secreting interleukin 2 is administered with another additive and/or cell lysis to prevent or treat cancer, particularly breast cancer (See, e.g., Deshmukh et al., 2001, J Neurosurg. 94:287 92). In other embodiments, gene therapy is conducted with the use of polynucleotide compounds, such as but not limited to antisense polynucleotides, ribozymes, RNA interference molecules, triple helix polynucleotides and the like, where the nucleotide sequence of such compounds are related to the nucleotide sequences of DNA and/or RNA of genes that are linked to the initiation, progression, and/or pathology of a tumor or cancer. For example, many are oncogenes, growth factor genes, growth factor receptor genes, cell cycle genes, DNA repair genes, and are well known in the art.

In another embodiment, the device and methods of the present invention are used in for administeration in conjunction with regimens of radiation therapy. For radiation treatment, the radiation can be gamma rays or X rays. The methods encompass treatment of cancer comprising radiation therapy, such as external beam radiation therapy, interstitial implantation of radioisotopes (I 125, palladium, iridium), radioisotopes such as strontium 89, thoracic radiation therapy, intraperitoneal P 32 radiation therapy, and/or total abdominal and pelvic radiation therapy. In preferred embodiments, the radiation treatment is administered as external beam radiation or teletherapy wherein the radiation is directed from a remote source. In various preferred embodiments, the radiation treatment is administered as internal therapy or brachytherapy wherein a radiaoactive source is placed inside the body close to cancer cells or a tumor mass. Also encompassed is the combined use the device and methods of the present invention with complexes and photodynamic therapy comprising the administration of photosensitizers, such as hematoporphyrin and its derivatives, Vertoporfin (BPD MA), phthalocyanine, photosensitizer Pc4, demethoxy hypocrellin A; and 2BA 2 DMHA.

In various embodiments, the device and methods of the present invention are used in association with at least one chemotherapeutic agent for the short treatment cycle of a cancer patient. The duration of treatment with the chemotherapeutic agent may vary according to the particular cancer therapeutic agent used. The invention also contemplates discontinuous administration or daily doses divided into several partial administrations. An appropriate treatment time for a particular cancer therapeutic agent will be appreciated by the skilled artisan, and the invention contemplates the continued assessment of optimal treatment schedules for each cancer therapeutic agent. The present invention contemplates at least one cycle, preferably more than one cycle during which a single therapeutic or sequence of therapeutics is administered. An appropriate period of time for one cycle will be appreciated by the skilled artisan, as will the total number of cycles, and the interval between cycles.

In another embodiment, the device and methods of the present invention are used in association with compounds that ameliorate the symptoms of the cancer (such as but not limited to pain) and the side effects produced complexes and combinations (such as but not limited to flu-like symptoms, fever, etc). Accordingly, many compounds known to reduce pain, flu-like symptoms, and fever can be used in combination or in admixture with the device and methods of the present invention. Such compounds include analgesics (e.g, acetaminophen), decongestants (e.g., pseudoephedrine), antihistamines (e.g., chlorpheniramine maleate), and cough suppressants (e.g., dextromethorphan).

### 5.4 Target Infectious Diseases

Infectious diseases that can be treated or prevented by the device and methods of the present invention are caused by infectious agents including, but not limited to, viruses, bacteria, fungi, protozoa, helminths, and parasites. The invention is not limited to treating or preventing infectious diseases caused by intracellular pathogens. Some of the commonly-used agents against infectious diseases and their appropriate doses and uses are known in the art and described in literature such as the Physician's Desk Reference (56^{th} ed., 2002).

Viral diseases that can be treated or prevented by in conjunction with the device and methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox.

Infectious virus of both human and non-human vertebrates, include retroviruses, RNA viruses and DNA viruses may be treated with the device and methods of the present invention. Examples of virus that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Retroviruses that are contemplated include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picomavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Many examples of antiviral compounds that can be treated with the device and methods of the present invention are known in the art and include but are not limited to: rifampicin, nucleoside reverse transcriptase inhibitors (e.g., AZT, ddI, ddC, 3TC, d4T), non-nucleoside reverse transcriptase inhibitors (e.g., Efavirenz, Nevirapine), protease inhibitors (e.g., aprenavir, indinavir, ritonavir, and saquinavir), idoxuridine, cidofovir, acyclovir, ganciclovir, zanamivir, amantadine, and Palivizumab. Other examples of anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime.

Bacterial infections or diseases that can be treated or prevented with the device and methods of the present invention are caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include but are not limited to infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, and Neisseria species. Specific examples of infectious bacteria include but are not limited to: Helicobacter pyloris, Borelia burgdorferi, Legionella pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Antibacterial agents or antibiotics that can be used in combination with the device and methods of the present invention include but are not limited to: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefininox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4 diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

Fungal diseases that can be treated or prevented by the device and methods of the present invention include but not limited to aspergilliosis, crytococcosis, sporotrichosis, coccidioidomycosis, paracoccidioidomycosis, histoplasmosis, blastomycosis, zygomycosis, and candidiasis.

Antifungal compounds that can be used in combination with the device and methods of the present invention include but are not limited to: polyenes (e.g., amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaflate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochloride.

Parasitic diseases that can be treated or prevented by the device and methods of the present invention including, but not limited to, amebiasis, malaria, leishmania, coccidia, giardiasis, cryptosporidiosis, toxoplasmosis, and trypanosomiasis. Also encompassed are infections by various worms, such as but not limited to ascariasis, ancylostomiasis, trichuriasis, strongyloidiasis, toxoccariasis, trichinosis, onchocerciasis. filaria, and dirofilariasis. Also encompassed are infections by various flukes, such as but not limited to schistosomiasis, paragonimiasis, and clonorchiasis. Parasites that cause these diseases can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

Many examples of antiprotozoal compounds that can be used in combination with the device and methods of the present invention to treat parasitic diseases are known in the art and include but are not limited to: quinines, chloroquine, mefloquine, proguanil, pyrimethamine, metronidazole, diloxanide furoate, tinidazole, amphotericin, sodium stibogluconate, trimoxazole, and pentamidine isetionate. Many examples of antiparasite drugs that can be used in combination with the present device and methods to treat parasitic diseases are known in the art and include but are not limited to: mebendazole, levamisole, niclosamide, praziquantel, albendazole, ivermectin, diethylcarbamazine, and thiabendazole. Further examples of anti-parasitic compounds include but are not limited to Acedapsone; Amodiaquine Hydrochloride; Amquinate; Arteflene; Chloroquine; Chloroquine Hydrochloride; Chloroquine Phosphate; Cycloguanil Pamoate; Enpiroline Phosphate; Halofantrine Hydrochloride; Hydroxychloroquine Sulfate; Mefloquine Hydrochloride; Menoctone; Mirincamycin Hydrochloride; Primaquine Phosphate; Pyrimethamine; Quinine Sulfate; and Tebuquine.

The present invention is useful in combination with a vaccine composition including without limitation a HSP or a α2M based vaccines. Examples of such vaccines for humans are described in The Jordan Report 2000, Accelerated Development of Vaccines, National Institute of Health, which is incorporated herein by reference in its entirety. Many vaccines for the treatment of non-human vertebrates are disclosed in Bennett, K. Compendium of Veterinary Products, 3rd ed. North American Compendiums, Inc., 1995, which is incorporated herein by reference in its entirety.

### 5.5 Therapeutic Regimens

For any of the combination therapies described above for treatment or prevention of cancer and infectious diseases, for use with the device and methods of the present invention, the combinations can be administered prior to, concurrently with, or subsequent to the administration of the other treatment non-HSP and non-α2M based modality. The non-HSP and non-α2M based modality can be any one of the modalities described above for treatment or prevention of cancer or infectious disease.

In one embodiment, treatments using the device and methods of the present invention can be administered to a subject at reasonably the same time as the other modality. This method provides that the two administrations are performed within a time frame of less than one minute to about five minutes, or up to about sixty minutes from each other, for example, at the same doctor's visit.

In another embodiment, treatments using the device and methods of the present invention are administered at exactly the same time. In yet another embodiment the treatments are administered in a sequence and within a time interval such that the treatment and the modality can act together to provide an increased benefit than if they were administered alone. In another embodiment, the treatments of the device and method of the present invention are administered sufficiently close in time so as to provide the desired therapeutic or prophylactic outcome. Each can be administered simultaneously or separately, in any appropriate form and by any suitable route. In one embodiment, the treatments of the device and methods of the present invention are administered by different routes of administration. In an alternate embodiment, each is administered by the same route of administration. The present invention can be used to administer treatments at the same or different sites, e.g. arm and leg. When administered simultaneously, the treatments may or may not be administered in admixture or at the same site of administration by the same route of administration.

In various embodiments, treatments utilizing the device and methods of the present invention are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In other embodiments, treatments utilizing the device and methods of the present invention and vaccine composition are administered 2 to 4 days apart, 4 to 6 days apart, 1 week a part, 1 to 2 weeks apart, 2 to 4 weeks apart, one moth apart, 1 to 2 months apart, or 2 or more months apart. In preferred embodiments, treatments utilizing the device and methods of the present invention are administered in a time frame where both are still active. One skilled in the art would be able to determine such a time frame by determining the half life of each administered component.

In one embodiment, treatments utilizing the device and methods of the present invention are administered within the same patient visit. In a specific preferred embodiment, treatments utilizing the device and methods of the present invention are administered prior to the administration of the modality. In an alternate specific embodiment, treatments utilizing the device and methods of the present invention are administered subsequent to the administration of the modality. In yet another specific embodiment, treatments utilizing the device and methods of the present invention are administered concurrently to the administration of the modality.

In certain embodiments, treatments utilizing the device and methods of the present invention are cyclically administered to a subject. Cycling therapy involves the administration one treatment for a period of time, followed by the administration of a modality for a period of time and repeating this sequential administration. Cycling therapy can reduce the development of resistance to one or more of the therapies, avoid or reduce the side effects of one of the therapies, and/or improve the efficacy of the treatment. In such embodiments, the invention contemplates the alternating administration of a complexes followed by the administration of a modality 4 to 6 days later, preferable 2 to 4 days, later, more preferably 1 to 2 days later, wherein such a cycle may be repeated as many times as desired. In other embodiments, treatments utilizing the device and methods of the present invention are alternately administered in a cycle of less than 3 weeks, once every two weeks, once every 10 days or once every week. In a specific embodiment, treatments utilizing the device and methods of the present invention are administered to a subject within a time frame of one hour to twenty four hours after the administration of a modality. The time frame can be extended further to a few days or more if a slow- or continuous-release type of modality delivery system is used.

### 5.6 Kits

The present invention also includes kits comprising the apparatus of the present invention. According to one embodiment, a kit of the present invention includes the apparatus of the present invention described above and also includes instructions for using the apparatus. In another embodiment, the kit of the present invention includes at least one aliquot of an appropriate additive such that a pharmaceutically acceptable composition for a predetermined medical or physical condition of a patient is included. According to yet another embodiment, a kit of the present invention includes a buffer. Another embodiment includes different needles in the kit, wherein the different needles can be for extracting different tissues, tissues from different depths, or tissues of different hardness. The different needles can also be for different functions, such as tissue extraction and the administration of the prepared treatment. Examples of such needles, by way of exmple but not limitation, include biopsy needles, stylet and cannula needles, hypodermic needles, and the like. In still another embodiment of the present invention, kits including different tissue lysing mechanisms, as described above, can be provided within a kit. In still yet another embodiment, kits can include a disposable apparatus and all the required attachments for utilizing the apparatus for a particular declaired procedure.

In a further embodiment, the kit comprises a unit dosage form of a pharmaceutical composition useful with the invention, e.g., an additive solution or treatment modality for use with the device and in the methods of the present invention.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

All references cited herein are incorporated by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

Furthermore, the foregoing descriptions of specific embodiments of the present invention are presented for purposes of illustration and description. They are not intended to be exhaustive or to limit the invention to the precise forms disclosed. Obviously many modifications and variations are possible in view of the above teachings. The embodiments were chosen and described above in order to best explain the principles of the invention and its practical applications, to thereby enable others skilled in the art to best utilize the invention and various embodiments with various modifications as are suited to the particular use contemplated. Furthermore, the order of steps in the method are not necessarily intended to occur in the sequence laid out. The invention may be embodied in other forms or carried out in other ways without departing from the spirit of the invention or the essential characteristics thereof. The present disclosure is therefore to be considered in all respects illustrative and not restrictive, the scope of the invention being indicated by the appended Claims, and all changes that come within the range and meaning of equivalency are intended to be embraced therein. In addition, any references cited above are incorporated herein by reference.

What we claim is:

1. A treatment device, comprising:
an extraction mechanism configured to extract a tissue sample;
a lysis mechanism coupled to the extraction mechanism, where said lysis mechanism is configured to induce cell lysis of said tissue sample to produce a lysed tissue sample; and
an administration mechanism coupled to both said extraction mechanism and said lysis mechanism, where said administration mechanism is configured to administer said lysed tissue sample to a patient.

2. The treatment device of claim 1, wherein said extraction mechanism comprises a syringe having a chamber adapted to couple with a collection needle.

3. The treatment device of claim 1, wherein said patient is a human.

4. The treatment device of claim 1, wherein said patient is an animal other than a human.

5. A treatment device, comprising:
an extraction mechanism configured to extract a tissue sample, where said extraction mechanism comprises a chamber adapted for coupling to a collection needle;
a lysis mechanism disposed within the chamber, where said lysis mechanism is configured to induce lysis of said tissue sample into a lysed tissue sample; and
an administration mechanism coupled to both said extraction mechanism and said lysis mechanism, where said administration mechanism is configured to administer said lysed tissue sample to a patient.

6. The treatment device of claim 2 or 5, wherein said chamber is coupled to said collection needle.

7. The treatment device of claim 2 or 5, wherein said extraction mechanism further includes a plunger configured to alter a pressure within said chamber of said extraction mechanism so as to extract said tissue sample and administer said lysed tissue sample.

8. The treatment device of claim 1 or 5, wherein said extraction mechanism is a biopsy device.

9. The treatment device of claim 8, wherein said biopsy device includes a stylet and cannula.

10. The treatment device of claims 1 or 5, wherein said lysis mechanism is selected from a group consisting of: a pair of rotatable cylinders, a pair of intermeshing rotatable gears, a grate, a tortuous path, rotatable blades, a cooling mechanism, a heat exchanger, an ultrasonic mechanism, an ultrasonic probe, and any combination of the aforementioned.

11. The treatment device of claim 2 or 5, wherein said administration mechanism comprises an administration needle in fluid communication with said chamber of said extraction mechanism.

12. The treatment device of claim 2 or 5, further comprising an additive mechanism in fluid communication with the chamber of said extraction mechanism, and configured to add an additive solution to said lysed tissue sample before said lysed tissue sample is administered to said patient.

13. The treatment device of claim 12, wherein said additive mechanism comprises a syringe in fluid communication with said chamber.

14. The treatment device of claim 1 or 5, wherein said extraction mechanism and said administration mechanism are the same mechanism.

15. A treatment device, comprising:
an extraction mechanism configured to extract a tissue sample, where said extraction mechanism includes a syringe type device having a chamber adapted for coupling to a collection needle and adapted for coupling to an administration needle;
a lysis mechanism disposed within the chamber, where said lysis mechanism is configured to induce lysis of said tissue sample into a lysed tissue sample; and
an administration mechanism coupled to both said extraction mechanism and said lysis mechanism, where said administration mechanism is configured to administer said lysed tissue sample to a patient, and where said administration mechanism at least partially includes said syringe type device.

16. The treatment device of claim 15, wherein said chamber includes said tissue sample selected from the group consisting of: a tumor sample; a tumor sample of a human, a tumor sample of an animal other than a human; a tumor sample that has been lysed and mixed with a fluid and homogenized; a tumor sample of a human that has been lysed and mixed with a fluid and homogenized; a tumor sample of an animal other than a human that has been lysed and mixed with a fluid and homogenized; an infected cell sample; an infected cell sample of a human; an infected cell sample of an animal other than a human; an infected cell sample that has been lysed and mixed with a fluid and homogenized; an infected cell sample of a human that has been lysed and mixed with a fluid and homogenized; and an infected cell sample of an animal other than a human that has been lysed and mixed with a fluid and homogenized.

17. A treatment device, comprising:
a lysis mechanism configured to induce cell lysis of a tissue sample; and
an administration mechanism removably coupled with said lysis mechanism and configured to administer said lysed tissue sample to a patient.

18. The treatment device of claim 17, further comprising an extraction mechanism configured to extract tissue from a subject, wherein said extraction mechanism is removably coupled with said lysis mechanism.

19. A method for treating a cancer, comprising:
extracting a tissue sample from a tumor into a chamber of a treatment device;
lysing said tissue sample into an lysed tissue sample within said chamber of said treatment device; and
administering said lysed tissue sample directly from said treatment device into a patient.

20. The method for treating a cancer of claim 19, further comprising, before said extracting, attaching a collection needle to said chamber.

21. The method for treating a cancer of claim 20, further comprising, before said extracting, inserting said collection needle into a tumor core of said patient.

22. The method for treating a cancer of claim 19, further comprising, before said administering, storing said lysed tissue sample.

23. The method for treating a cancer of claim 19, wherein said lysing comprises blending, grating, crushing, thermal treating, or sonication of said tissue sample.

24. The method for treating a cancer of claim 19, wherein said lysing comprises:
cooling said tissue sample to at least −196 degree Celsius for between five seconds to ten minutes; and
warming said tissue sample to approximately 37 degrees Celsius for between five seconds to ten minutes.

25. The method for treating a cancer of claim 19, wherein said lysing comprises:
cooling said tissue sample chamber with liquid nitrogen for between approximately five seconds to ten minutes; and
warming said tissue sample by subjecting said tissue sample chamber to a water bath at between approximately 37 degrees Celsius for between five seconds to ten minutes.

26. The method for treating a cancer of claim 19, further comprising, before said administering, adding an additive solution to said lysed tissue sample.

27. The method for treating a cancer of claim 26, wherein said additive solution is selected from the group consisting of one or more of: a cytokine, an adjuvant, an antibody, a biological response modifier, an agonist of a ligand, or an antagonist of a ligand, a receptor, or a signal transduction molecule of the immune system, an anticancer agent, and any combination of the aforementioned.

28. The method for treating a cancer of claim 26, wherein said additive solution is selected from the group consisting of one or more of: a saponin adjuvant, a heat shock protein (HSP), an complex of HSP-antigenic peptide complex, a complex of antigenic molecules, a A2Malhpa 2 macroglobulin, a lipopolysaccharide (LPS)n, an immunostimulatory oligonucleotide, an anti-4-1BB antibody, an anti-CTLA4 antibody, an anti-OX40, and any combination of the aforementioned.

29. The method for treating a cancer of claim 19, further comprising, before said administering, replacing a collection needle with an administration needle.

30. The method for treating a cancer of claim 19, further comprising, before said administering, inserting an administration needle of said treatment device into said patient at a different location to where said extracting occurred.

31. The method for treating a cancer of claim 19, wherein said tissue sample is a tumor core.

32. A method for treating a cancer, comprising:
attaching a collection needle to a chamber of a treatment device;
inserting said collection needle into a tumor;
extracting a tissue sample from said tumor into said chamber of said treatment device;
lysing said tissue sample into a lysed tissue sample within said chamber of said treatment device;
adding an additive solution to said lysed tissue sample;
replacing said collection needle with an administration needle;
inserting said administration needle of said treatment device into a patient at a different location to where said extracting occurred; and
administering said lysed tissue sample directly from said treatment device into said patient.

33. A treatment method, comprising:
extracting a tissue sample from a patient into a treatment device;
lysing said tissue sample into an lysed tissue sample within a chamber of said treatment device; and
administering said lysed tissue sample directly from said treatment device into the patient to produce an immune response.

34. A treatment method, comprising:
lysing a tissue sample into a lysed tissue sample within a chamber of a treatment device; and
administering said lysed tissue sample directly from said treatment device into a patient such that an immune response is produced.

35. The treatment method of claim 34, wherein said tissue sample is obtained from the group consisting of the patient, a human other than the patient, a mammal, and a tissue culture.

36. The treatment method of claim 34, wherein said immune response is directed toward an infectious disease or a cancer.

37. A kit for a device for producing an immune response, comprising:
a lysis mechanism configured to induce cell lysis of a tissue sample;
an administration mechanism coupled to said lysis mechanism, wherein said administration mechanism is configured to administer said lysed tissue sample to a patient; and
instructions for using said device.

38. The kit of claim 37, further comprising an extraction mechanism configured to extract tissue from a subject.

39. The kit of claim 38, wherein said extraction mechanism includes a collection needle, a biopsy needle, a stylet, or a cannula.

40. The kit of claim 37, wherein said administration mechanism includes an admininistration needle.

41. The kit of any one of claims 37 to 40, further comprising at least one biologically active additive.

42. The kit of claim 41, wherein said biologically active additive is selected from the group consisting of one or more of: a cytokine, an adjuvant, an antibody, a biological response modifier, an agonist of a ligand, or an antagonist of a ligand, a receptor, or a signal transduction molecule of the immune system, an anticancer agent, an anti-infective agent and any combination of the aforementioned.

43. The kit of any one of claims 37 to 42, further comprising a buffer.

44. The kit of any one of claims 37 to 43, further comprising a tissue sample.
